(19) United States
(12) Patent Application Publication
(10) Pub. No.: US 2023/0321459 A1
(43) Pub. Date: Oct. 12, 2023

(54) METHODS AND SYSTEMS FOR OPTIMIZING VOLUMETRIC MODULATED ARC THERAPY (VMAT) TREATMENT PLANS

(71) Applicant: Health Research, Inc., Buffalo, NY (US)

(72) Inventor: Daryl NAZARETH, Buffalo, NY (US)

(21) Appl. No.: 18/040,670

(22) PCT Filed: Aug. 6, 2021
(86) PCT No.: PCT/US21/45123

§ 371 Date: Feb. 6, 2023

Related U.S. Application Data
(60) Provisional application No. 63/062,388, filed on Aug. 6, 2020.

(51) Int. Cl. A61N 5/10

(52) U.S. Cl. CPC A61N 5/1031; A61N 5/1036; A61N 5/1047; A61N 2005/1034

(57) ABSTRACT

A volumetric modulated arc therapy (VMAT) treatment plan may be optimized by obtaining a VMAT treatment plan and calculating a radiation dose matrix corresponding to each a plurality of beamlets, wherein each beamlet represents a change in field when an MLC leaf is moved a predetermined unit distance. The method includes defining an enhanced objective function (EOF) for achieving one or more clinical objectives and minimizing the EOF for proposed leaf positions iterating through each leaf of at least a subset of the leaves of the VMAT treatment plan (wherein the proposed leaf positions move each leaf into the field or out of the field by the predetermined unit distance and correspond to the addition or subtraction of the corresponding radiation dose matrix). The set of leaf positions of the VMAT treatment plan is updated according to the proposed leaf positions of the minimized EOF.

### CROSS-REFERENCE TO RELATED APPLICATIONS

This application claims priority to U.S. Provisional Application No. 63/062,388, filed on Aug. 6, 2020, now pending, the disclosure of which is incorporated herein by reference.

### FIELD OF THE DISCLOSURE

The present disclosure relates to volumetric-modulated arc therapy planning.

### BACKGROUND OF THE DISCLOSURE

According to a study by World Health Organization, approximately 10.0 million deaths have been reported worldwide among 19.3 million new diagnosed cancer cases in 2020. About half of cancer patients receive radiation therapy (RT) to treat tumors. Cutting-edge technology utilizing megavoltage linear accelerators and advanced treatment planning systems (TPS) make RT a frontline method for cancer treatment. The TPS used at Roswell Park Comprehensive Cancer Center, and one in widespread use throughout the United States, is Eclipse (Varian Medical System, Palo Alto, CA).

One of the most common RT treatment methods is Volumetric Modulated Arc Therapy (VMAT), in which the dose delivery involves several dynamic parameters, e.g., couch, collimator and gantry angles, gantry speed, dose rate and collimator position. The parameters are typically grouped into 178 segments or control points (CPs) per delivery arc (FIG. 1). Initial treatment objectives are defined clinically, with specific dose-volume coverage for the target and organs at risk (OARs). This permits an inverse planning approach, with the aim of delivering a uniform prescription dose to the tumor, while ensuring the OAR doses remain below the clinical objective values. VMAT inverse planning employs an optimization algorithm, such as gradient descent, along with a rapid dose calculation method, such as pencil beam calculation, to evaluate the objective function. The optimization process determines the CP parameters to be used in the VMAT treatment. Once the optimization step is complete, a final, accurate dose calculation is performed to provide the actual deliverable dose distribution. However, since the pencil beam dose calculator used during optimization is not accurate, the optimized VMAT plan may not occupy a true local minimum in objective function space. That is, the plan may potentially be improved (i.e., the objective score reduced) by searching its local neighborhood, as shown schematically in FIGS. 2A and 2B.

### BRIEF SUMMARY OF THE DISCLOSURE

The present disclosure provides approaches to optimize VMAT treatment plans. In a first aspect, an enhanced optimization (EO) employs the TPS VMAT plan as a starting point, and applies small perturbations to nudge the solution closer to a true objective minimum. The perturbations are comprised of beamlet dose matrices, calculated using Monte Carlo routines on a distributed-computing framework. This permits the objective space in the neighborhood of the TPS plan to be explored for locations of lower minima. Since the beamlets are calculated using an MC dose calculation algorithm, the scores computed during the EO search are more accurate than those computed by the TPS optimizer. The resulting plan is then imported into the TPS in order to determine the final, deliverable dose, and to compare the EO and original plans.

In another aspect, a weight/intensity-level linear optimization is provided wherein weights, or intensities, of each CP are used as variables. The starting value of each weight is given by the treatment planning system as the meterset. By varying each meterset, the contribution from each CP can be increased or decreased. This provides the advantage of making each variable continuous, rather than discrete, and therefore amenable to any continuous-variable optimization algorithm. In addition, the dose matrix for each CP is only required to be calculated once, since varying the weight is equivalent to multiplying the matrix by a single scale factor (a separate factor for each CP). As the weights are varied, the new dose is calculated by summing the individual CP dose matrices. The objective function is evaluated, and optimization proceeds iteratively until a (possibly local) minimum is located.

### DESCRIPTION OF THE DRAWINGS

For a fuller understanding of the nature and objects of the disclosure, reference should be made to the following detailed description taken in conjunction with the accompanying drawings, in which:

FIG. 1: A schematic of a set of control points (CPs) for a volumetric modulated arc therapy (VMAT) arc. The grey box shows multileaf collimator (MLC) settings for one CP.

FIG. 2A: A simplified 1-dimensional (1-D) representation of the objective space for a VMAT optimization problem. The grey curve is the score calculated by Eclipse during optimization, while the black curve is the true score. After Eclipse locates an approximate local minimum, it is used as the starting point for the presently-disclosed enhanced optimization (EO). The EO searches the neighborhood using a more accurate dose (and therefore score) calculator.

FIG. 2B: An enlargement of the region of FIG. 2A indicated by the grey box. Note that FIGS. 2A and 2B are illustrative and is not based on actual, calculated values.

FIG. 3: A flow chart of the clinical treatment planning process. The conventional workflow is indicated in black, while the proposed EO addition is indicated in grey. The EO calculations currently run 10-20 hours, but are mostly executed by scripts, and may be performed overnight.

FIG. 4: The beam's eye view of contoured patient anatomy for a prostate VMAT plan at control point 55. The grey square at leaf A27 is the cross section of a beamlet generated by moving the leaf out by 0.5 cm.

FIG. 5: An axial cross section of the same anatomy shown in FIG. 4 with the beamlet dose displayed.

FIG. 6: Perturbations to the treatment planning system (TPS) plan are performed by adding/subtracting a radiation dose matrix to/from the current dose matrix, depending on whether the MLC leaf position is moved out of or into the field.

FIG. 7: Dose-volume histogram (DVH) curves for the first brain VMAT plan before (original) and after EO. The pituitary planning risk volume (PRV) improved, with a small increase to the 59.4 planning target volume (PTV). Organs at risk (OAR's) with objectives already met before and after EO are not shown.

FIG. 8: DVH curves for the second brain VMAT plan. The cochlea, lacrimal, lacrimal PRV, and pituitary were improved by the EO. Note that the lacrimal and lacrimal PRV dose objectives are identical, and are very close to the right cochlea PRV objective.

FIG. 9: DVH curves for the pediatric brain VMAT plan. There was a slight improvement to the left cochlea PRV.

FIG. 10: DVH curves for the first head and neck VMAT plan. There was an improvement to the right cochlea and a small increases in both PTV doses.

FIG. 11: DVH curves for the second head and neck VMAT plan. Improvements to the trick planning structures (Rings) produced improvements to the left cochlea.

FIG. 12: DVH curves for the first prostate VMAT plan, showing very little change with the EO. Note that the max dose objective for the bladder, rectum, and penile bulb are identical. The left and right femoral head dose objectives are also identical.

FIG. 13: DVH curves for the second prostate VMAT plan, showing no change with the EO. Note that the femoral head objectives are identical.

FIG. 14A: DVH's for the pediatric brain case comparing the EO and the TPS plans using the Type 2 objectives.

FIG. 14B: A magnification of the DVH for the left cochlea PRV of FIG. 14A.

FIG. 15: DVH curves for the pediatric brain case after the EO was applied. “No Mod” indicates the original objectives, while “Type 1” and “Type 2” objectives are progressively stricter. Type 1 plan was the same as the No Mod plan, as explained in Discussion.

FIG. 16A: Isodose curves for the pediatric brain case, showing the left cochlea PRV, which is shaded. The no-modification and Type 1 plans are identical.

FIG. 16B: Isodose curves for the pediatric brain case of FIG. 16A, wherein the Type 2 plan shows sparing of the left cochlea PRV. Note that the isodose curves indicate a lower dose toward the center of the structure.

FIG. 17: DVHs for a head and neck VMAT case, comparing the TPS, EO, and EO-water plans. The EO resulted in the lowest score.

FIG. 18: An enlargement of the rectum and PTV DVH curves for the prostate case after various modifications were made to the objectives. No Mod indicates the original objectives, while Type 1 and Type 2 objectives are progressively stricter. The Type 3 dose objectives were the same as Type 2, but with reduced priority weights. In every plan, reduction of the rectum dose resulted in compromised PTV dose homogeneity.

FIG. 19A: DVH curves for the same dose matrices calculated using the same plan parameters before and after EO by MC.

FIG. 19B: DVH curves for the same dose matrices calculated using the same plan parameters before and after EO by Eclipse for comparison with FIG. 19A. Differences are especially apparent for the rectum and penile bulb. Note that the max dose objective for the bladder, rectum, and penile bulb are identical. The left and right femoral head objectives are also identical.

FIG. 20: The Monte Carlo (MC)-calculated DVH curves of the pediatric brain case's left cochlea PRV resulting from the original EO, type 1, and type 2 plans. The DVH's for the original EO and type 1 plans are identical, and the type 2 plan's DVH coincides with the other two at doses greater than about 33.1 Gray (Gy). Thus, the region under all three curves evaluated using the original and type 1 objective functions are identical and produce the same penalties.

FIG. 21: A chart showing a Meterset Weight of 178 CPs for each of two arcs that comprise an exemplary plan.

FIG. 22: A chart depicting a VMAT Treatment Plan optimization method according to an embodiment of the present disclosure.

FIG. 23: A chart depicting another VMAT Treatment Plan optimization method according to another embodiment of the present disclosure.

FIG. 24: A chart depicting a system for VMAT Treatment Plan optimization according to another embodiment of the present disclosure.

### DETAILED DESCRIPTION OF THE DISCLOSURE

Volumetric modulated arc therapy (VMAT) is a radiation treatment delivery modality based on inverse planning, a process which commonly employs dose-volume objectives for the planning target and organs at risk (OAR's). These objectives are used to define an objective function as a function of the VMAT treatment parameters. A widely-used objective function is Eq. (1) where w_{D} is the set of parameters (e.g., multileaf collimator (MLC) leaf positions) for the plan. This function quadratically adds the dose objective violations, d_{j,min}−dᵢ(w_{D}) for minimum dose objectives and dᵢ(w_{D})−d_{j,max} for maximum dose objectives, over individual voxels, i, which violate dose objectives, d_{j,max} or d_{j,min}, and then sums all of the penalties into a single score. The Heaviside function (Eq. (2)) limits the penalty to the range of doses which violate the objective. Wⱼ is a priority weighting given to individual objectives. If a calculated dose-volume meets an objective, no penalty is incurred.

$$f ( {w}_{D} ) = {\sum }_{j} {\sum }_{i} {W}_{j} [ {( {d}_{j , min} - {d}_{i} ( {w}_{D} ) )}^{2} * H ( {d}_{j , min} - {d}_{i} ( {w}_{D} ) ) + {( {d}_{i} ( {w}_{D} ) - {d}_{j , max} )}^{2} * H ( {d}_{i} ( {w}_{D} ) - {d}_{j , max} ) ] \qquad ( 1 )$$

$$H ( d ) = \{ \begin{matrix}1 & d \geq 0 \\ 0 & d < 0\end{matrix} \qquad \text{(2)}$$

The treatment planning system (TPS) typically applies an optimization algorithm to minimize the function ƒ(w_{D}). During this process, the dose distribution to the patient is calculated every time ƒ(w_{D}) is evaluated. Because this calculation occurs a large number of times, it is performed very rapidly, typically within the time frame of milliseconds. Eclipse Version 15.6 (Varian Medical Systems, Palo Alto, CA) employs a pencil beam dose calculation algorithm, which is relatively inaccurate, to determine dose while optimizing. Once the optimization process is complete, an accurate dose calculation (using Acuros or the Anisotropic Analytical Algorithm (AAA)) is performed to provide the final, deliverable dose distribution.

### Enhanced Optimization

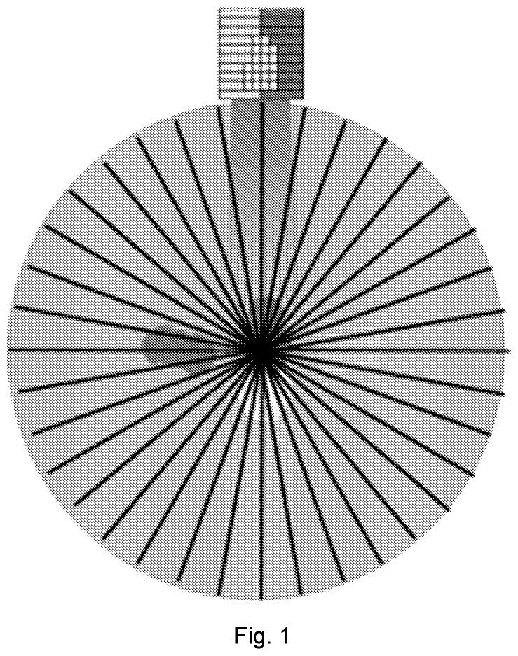

Since the TPS dose algorithm used during optimization is inaccurate, it does not necessarily model the objective function landscape correctly. Even in the case of Eclipse's Progressive Resolution Optimization (PRO) algorithm, which periodically performs an accurate dose calculation during optimization, the majority of the dose calculation is performed using the faster pencil beam technique. Therefore, the function which is minimized may be substantially different from the true objective function, which would be obtained with a more accurate dose algorithm. This results in the function minima, whether local and global, being inaccurately identified even with an effective optimization algorithm and an accurate final, deliverable dose calculation. This situation is represented schematically in FIG. 1 (note that the graph is meant to illustrate the differences between the TPS and true values, and does not represent actual calculations). The present disclosure improves on the optimization process by beginning with the TPS-optimized plan, which lies in an approximate local minimum, and searching this neighborhood for a plan whose score would be lower when calculated with an accurate dose algorithm. Much work has been done in evaluating and improving VMAT optimization algorithms. To the best of our knowledge, however, there has been no investigation into this type of post-optimization VMAT improvement.

For this work, Enhanced Optimization (EO) is defined as the process of making further refinements the MLC leaf positions of a VMAT plan which has been optimized by a TPS. This concept was investigated for intensity-modulated radiation therapy (IMRT) plan optimization by Niu et al. Niu used simple beamlet calculations to estimate perturbations to an IMRT plan's dose by moving MLC leafs in or out by 0.5 cm, a process referred to as post-optimization refinement (POpR). It was found that, by employing a greedy search algorithm, an IMRT plan could be improved quickly. However, an analogous process for VMAT involves a much larger number of control points and beamlets. In addition, the presently-disclosed techniques utilizes a more accurate method for computing beamlet dose matrices thereby further improving the quality of the resulting plan.

### Monte Carlo Calculations

Monte Carlo (MC) simulation is a stochastic dose computational method that determines the behavior of a macroscopic system by averaging microscopic events, or histories. These histories include particle interactions and their associated probabilities. The American Association of Physicists in Medicine (AAPM) Task Group (TG) Report indicates that MC methods are more accurate than the conventional dose calculation algorithms employed by typical TPS's. This is particularly true in heterogeneous media, where the accurate modeling of electron transport is especially challenging. In one study, MC calculations were used, together with a direct-aperture optimization algorithm, to create IMRT plans in heterogeneous low-density media (e.g., lung tissue).

Typically, secondary monitor unit (MU) verification is performed with dose algorithms which are less accurate that those of the TPS. However, due to advances in computing techniques, TG-114 recommends more sophisticated algorithms for this purpose. A study compared MC VMAT calculations, employing vendor-provided phase space data, to TPS calculations, and found agreement to within 2% in high-dose regions. Another study involved comparison of an MC method to Eclipse's AAA for VMAT calculations, and found absolute dose value agreement to within 3%. However, these studies compared the deliverable VMAT plan doses. Although it is difficult to evaluate directly, the pencil-beam algorithm used by Eclipse during the optimization process will be much less accurate than the MC or AAA methods. A well-known trade-off with the high accuracy of MC methods is their large computational costs. They require extensive working memory and long execution times. Therefore, a complete optimization process using MC is impractical. However, we may use MC calculations to provide perturbations to a VMAT plan after it has been optimized by the TPS. These perturbations involve beamlet dose calculations, which may be performed by an MC method in a reasonable time frame (see Materials and Methods below).

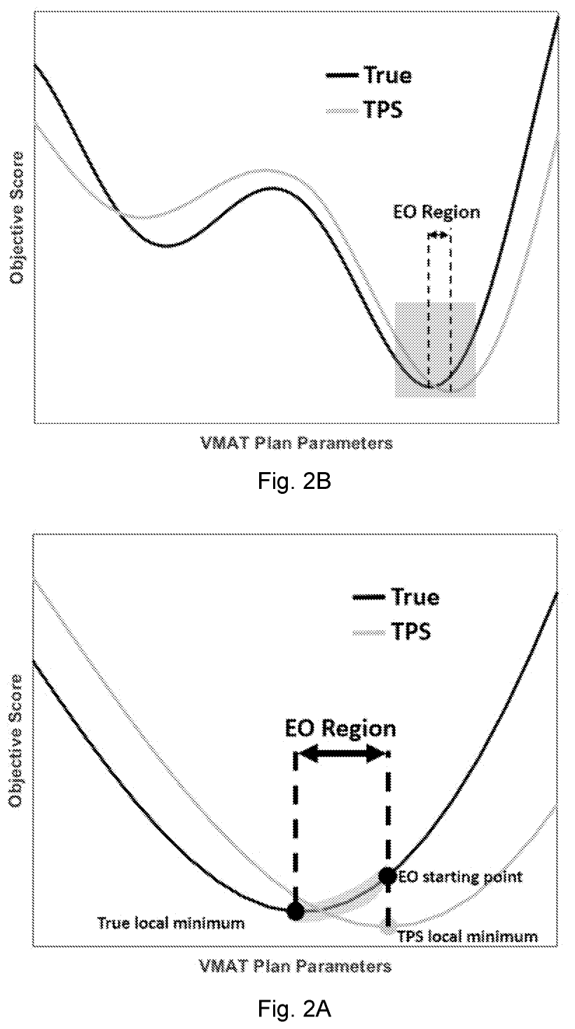

FIGS. 2A and 2B shows a simplified representation of both the true solution space and the space calculated by the TPS during VMAT plan optimization. The objective function value is sketched against the VMAT plan parameters. Since the TPS employs an inaccurate pencil beam algorithm during this process, its score (grey curve) is only an approximation of the true score (black curve). Note that the black curve is an idealized function, because it is not possible in practice to calculate the true objective function exactly. However, MC simulations are the gold standard in dose calculation accuracy, and therefore will closely match the true values. The TPS creates the VMAT plan by locating a minimum (local or possibly global) of the grey curve. The Enhanced Optimization process uses those VMAT parameters as a starting point, performs a search in its neighborhood using MC calculations, and attempts to locate a nearby minimum of the black curve. This neighborhood is indicated in the figure as EO Region.

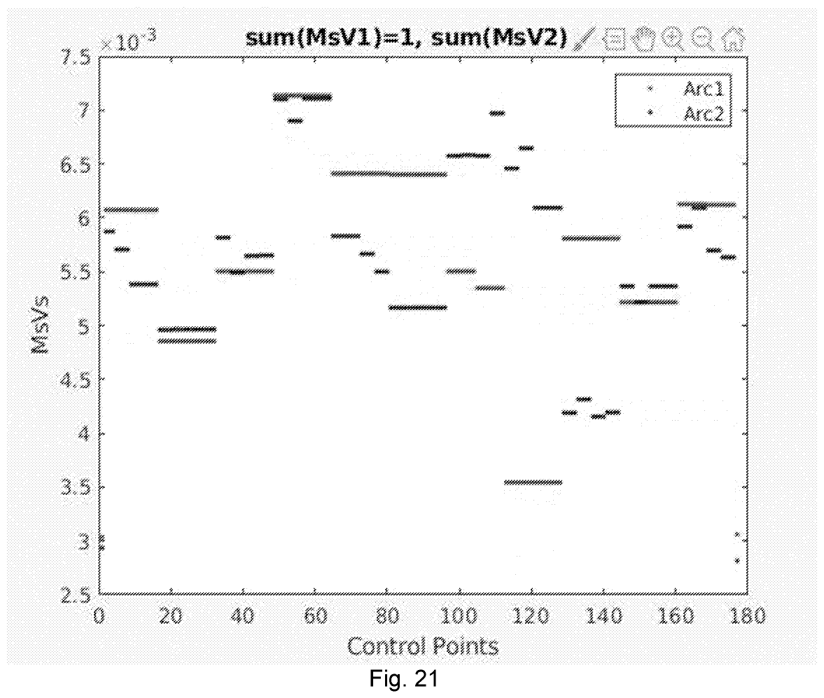

With reference to FIG. 22, in a first aspect, the present disclosure may be embodied as a method 100 for optimizing a volumetric modulated arc therapy (VMAT) treatment plan. The method 100 includes obtaining 103 a VMAT treatment plan from a treatment planning system (TPS). The VMAT treatment plan includes a plurality of control points, each control point having a set of leaf positions corresponding to a set of leaves of a multileaf collimator (MLC) in a field of a linear accelerator (linac).

A radiation dose matrix is calculated 106 for each beamlet a radiation dose matrix corresponding to each beamlet. A beamlet is the change in field when an MLC leaf is moved a predetermined unit distance. The beamlet dose matrices may be calculated using Monte Carlo routines. In other words, for each desired leaf movement (of a unit distance) into the field or out of the field, a radiation dose matrix is calculated. A radiation dose matrix may also be calculated for not movement for each leaf. The desired leaf movements may include a subset of the set of leaves of the MLC. For example, the desired leaf movements may only include the subset of “active” leaves—e.g., leaves that effect the target volume. Other subsets may be used. For example, it may be determined that the leaves of a sub-arc of the linac have the highest likelihood of improving a treatment plan for a particular target volume (e.g., a head-and-neck, a rectum, etc.)

The method 100 includes defining 109 an enhanced objective function (EOF) for achieving one or more clinical objectives, including achieving at least a minimum dose to a target volume and minimizing a dose to an organ at risk. There may be more than one target volumes and/or more than one OARs included within the scope of an EOF within the present scope. The EOF may be the same as the objective function used to generate the original VMAT treatment plan (e.g., generated by the TPS). In some embodiments, the EOF is different from the objective function used to generate the original VMAT treatment plan. The EOF is minimized 112 for proposed leaf positions iterating through each of at least a subset of the leaves of the VMAT treatment plan (e.g., the subset of desired leaves as described above, such as, for example, the active leaves, etc.) In some embodiments, the at least a subset includes all of the leaves of the VMAT treatment plan. The proposed leaf positions move each leaf into the field or out of the field by the predetermined unit distance and corresponds to the addition or subtraction of the corresponding radiation dose matrix. For example, when a leaf is moved into the field, the corresponding dose matrix is subtracted.

The proposed leaf position of each leaf may be represented by a vector (x) of ternary leaf variables, and the EOF (ƒ_{E}) is a function of the vector (ƒ_{E}(x)). As further described below under the heading Materials and Methods.

The set of leaf positions of the VMAT treatment plan is updated 115 according to the proposed leaf positions of the minimized EOF. The VMAT treatment plant updated in this way may be considered a new treatment plan—i.e., an EO treatment plan. The minimizing and updating steps may be performed for each control point of the VMAT treatment plan.

In some embodiments, the method 100 includes recalculating 118 the updated VMAT treatment plan with linac constraints and/or leaf-motion constraints. For example, the updated VMAT treatment plan may be recalculated on the TPS in order to obtain a final plan. In some embodiments, DVHs and/or isodose curves of the updated VMAT treatment plan may be generated 121. Such reports may be compared to those of the original VMAT treatment plan so as to decide on the appropriate plan (as further described below).

### Materials and Methods

### Exporting VMAT Plan Files

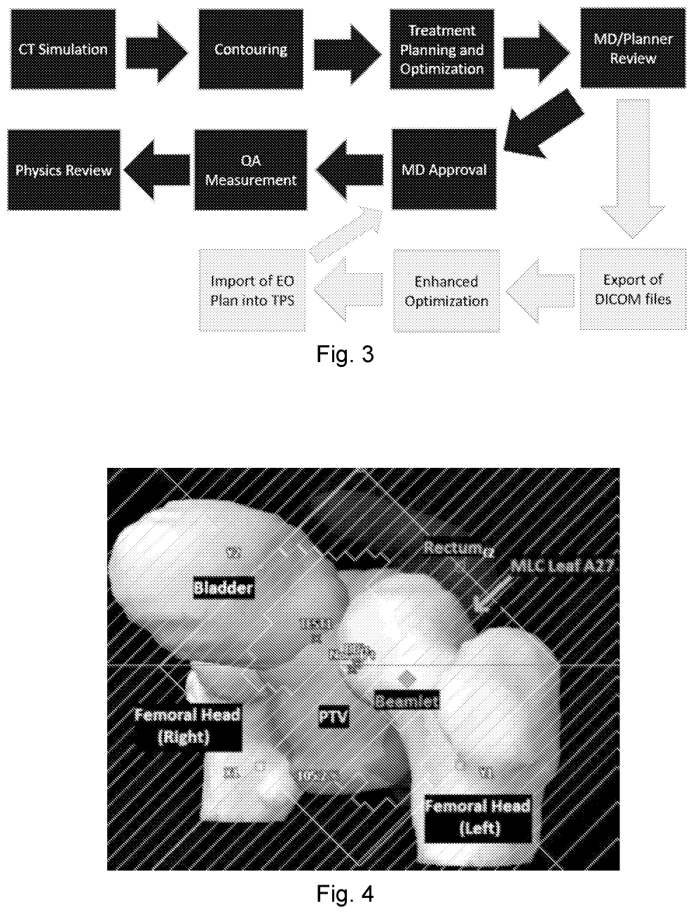

In a typical clinical work flow, the VMAT plan is created and optimized by a planner and then approved by a physician. The presently-disclosed EO can be performed before or after physician approval, and the resulting plan can be compared with the original for final approval. FIG. 3 illustrates this exemplary workflow. The EO process begins with a VMAT plan which has been optimized by the TPS. In addition to computed tomography (CT) data, three DICOM files are exported: the dose file containing the dose matrix and corresponding DVH's, the plan file containing the control point information, and the structures file containing the contoured structure data.

TABLE 1
VMAT plans used in evaluations of the EO

| Plan | Arcs | Objectives | Variables |
|---|---|---|---|
| Brain 1 | 2 | 17 | 2733 |
| Brain 2 | 2 | 23 | 3831 |
| Ped. Brain | 2 | 19 | 2714 |
| H&N 1 | 2 | 16 | 4548 |
| H&N 2 | 2 | 17 | 2974 |
| Prostate 1 | 2 | 17 | 5099 |
| Prostate 2 | 2 | 8 | 3234 |

TABLE 2
Objective modification types used for the pediatric brain case.
The objective is the maximum dose to the left cochlea PRV.

| Objective | Maximum |
|---|---|
| Unmodified | 35.0 Gray (Gy) |
| Type 1 | 33.5 Gy |
| Type 2 | 29.0 Gy |

### Beamlet Calculation Using Monte Carlo

The Electron Gamma Shower, National Research Council (EGSnrc) toolkit is used for all MC dose calculations performed outside of the TPS. The BEAMnrc module is used to model the linear accelerator (linac), and the DOSXYZnrc module is used to create the patient/phantom model.

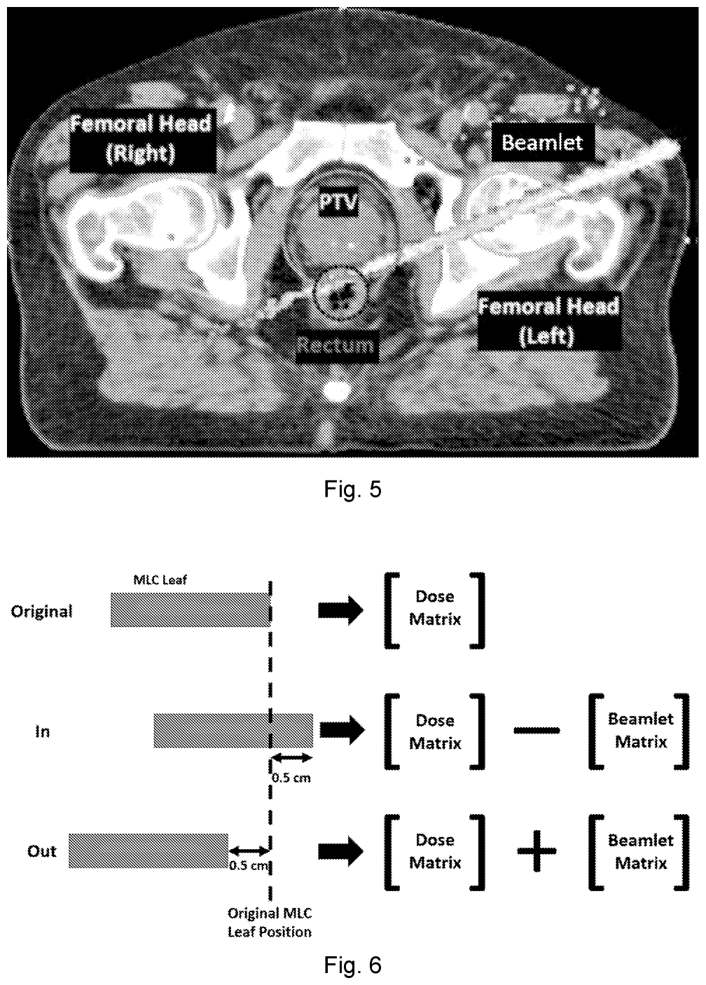

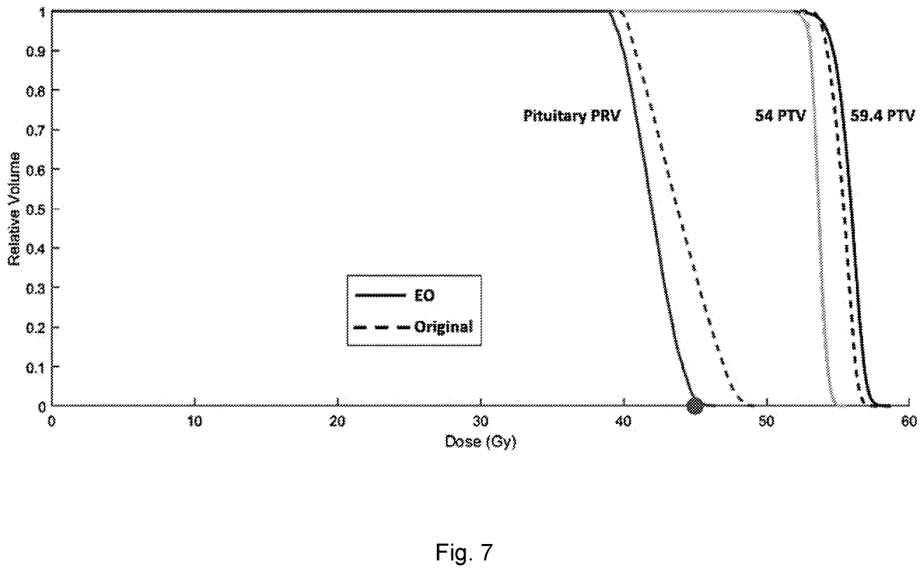

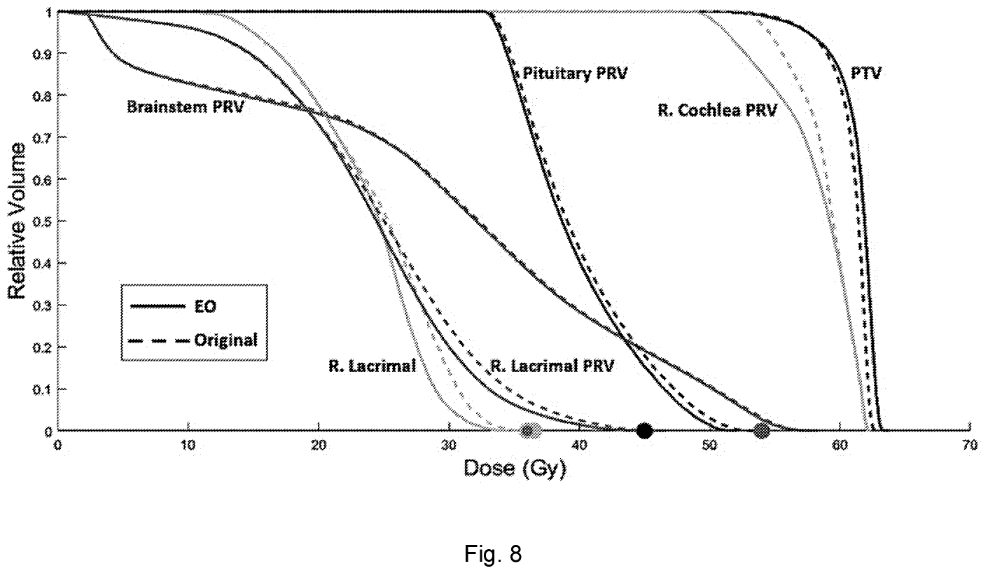

Perturbations to the original matrix are created by simulating beamlets. A beamlet is a change in dose resulting from moving a given MLC leaf into or out of the field by a small step (a predetermined unit distance), for a given control point. In this study, the step size used was 0.5 cm, although this value may be modified (see Discussion). FIG. 4 shows the beam's eye view of a beamlet for a prostate plan, while FIG. 5 shows a transverse view of this beamlet. Therefore, each beamlet corresponds to a small dose matrix, which can be calculated by simulating the modified VMAT plan (original plan, except with a single leaf moved in or out, in a single control point), and subtracting the original dose matrix. In practice, this may be accomplished using partial arcs, each comprising three adjacent control points. Any set of MLC perturbations can then be modeled by considering the original dose matrix and then adding or subtracting the corresponding beamlet matrices. This process is illustrated in FIG. 6.

A typical VMAT plan includes 2-4 arcs, each with 178 control points (which may be, for example, 2° per control point, representing arcs of 356°), with each control point involving 10-40 leaves. Therefore, there is a large number of beamlets associated with a VMAT plan. This number may be reduced by only considering the beamlets that irradiate the target structures. In addition, leaf motion that would violate leaf speed and collision constraints is excluded. The resulting number of beamlets is generally 5,000-10,000. For this study, the MC beamlet calculations were parallelized on a computer cluster. This allowed simulations to be run in batches of 500, with further parallelization possible over multiple processing cores. The time for a complete set of beamlet calculations (corresponding to one VMAT plan) was approximately 10 to 20 hours. The calculated beamlet matrices are saved to a library, for use in the greedy search described below.

### Enhanced Optimization Objective Function

The original TPS dose distribution is modified by adding or subtracting beamlets, which corresponds to moving leaves out of or into their fields, respectively. This permits a new enhanced objective function (EOF) to be defined:

$${f}_{E} ( x ) = {\sum }_{j} {\sum }_{i} {W}_{j} [ \text{} {( {d}_{j , min} - {d}_{i} ( x ) )}^{2} * H \{ {d}_{j , min} - {d}_{i} ( x ) \} + {( {d}_{i} ( x ) - {d}_{j , max} )}^{2} * H \{ {d}_{i} ( x ) - {d}_{j , max} \} ] \qquad ( 3 )$$

This is similar to Eq. (1), but where:

x=[x₁,x₂, . . . ,xₙ] (4)

is a vector of ternary leaf variables, and n is the number of active leaves in the process. That is, each of the xᵢ∈{−1, 0, 1} represents a possible leaf position modification. A −1 indicates a leaf moving into the field (i.e., the subtraction of a beamlet from the total dose matrix); a 0 indicates no change; and a 1 indicates a leaf moving out of the field (addition of a beamlet to the total dose matrix). Therefore,

S≡ƒ_{E}(x₀), (5)

where x₀=[0, 0, . . . , 0] is the value of the EOF applied to the original TPS-optimized plan (i.e., with no changes to the leaf positions). The EOF is a function of ternary, and thus discrete, variables, and therefore it may be optimized using a discrete optimization algorithm (see Discussion). In this study, we performed a simple greedy search, which iterated through each active leaf, one at a time, and calculated the EOF corresponding to the leaf moving in or out. A leaf position was saved if it resulted in a reduction (improvement) in EOF value, and rejected otherwise. In other words, we first check if

ƒ_{E}(x′)<ƒ_{E}(x),for x=x₀and x′=[1,0, . . . ,0] or [−1,0, . . . ,0]. (6)

If that condition is met, we replace x by x′ and check if

ƒ_{E}(x′)<ƒ_{E}(x),for x′=[0,1, . . . ,0] or [0,−1, . . . ,0]. (7)

This process iterates over every active leaf (i.e., 1 to n). The average run time of the greedy search was 30-45 minutes using the available compute cluster; this is dependent on the number of active leaves and number of objectives in the EOF.

### Evaluations

This study used retrospective comparisons in which actual clinical VMAT plans were subject to the Enhanced Optimization process. Seven VMAT plans (two adult brain, one 18-year-old pediatric brain, two head and neck, and two prostate) were selected at random from the Eclipse database. Table 1 shows information on the number of objectives and ternary EO variables of each plan. A standard evaluation was performed on every plan, employing the same OAR dose-volume objectives as in the TPS plans. In order to investigate further potential improvements, the pediatric brain plan was subject to additional evaluations in which the left cochlea PRV objective was reduced (i.e., made stricter). This objective was selected because it was the only one not satisfied in the original TPS plan. The objective was modified to 29 Gray (Gy) from 35 Gy; the plan was optimized in Eclipse, and then the EO was applied. In addition, the EO was applied to the original Eclipse plan, with the left cochlea PRV modified by various amounts. These specific objectives are shown in Table 2. In all cases, the PTV objectives were modified because the original planning required certain optimization strategies, which were not required in the EO process. When performing VMAT planning with Eclipse, a frequent strategy is to increase the minimum-dose objectives to target structures by 2-4%. This practice tends to result in the correct minimum dose in the final plan, and was followed for the target objectives used in this study.

For each case, the results of the EO were used to modify the control point information in the plan DICOM file, which was then imported into Eclipse. This allowed the plan to be recalculated with any linac and leaf-motion mechanical constraints applied. The EO plan could then be compared to the original Eclipse plan by reviewing DVH's and isodose curves. To simplify the comparison, the EO plan was normalized so that 95% of the PTV received the same dose as in the corresponding Eclipse plan. The final Eclipse and EO dose matrices were also exported in order for their objective scores to be calculated.

An interesting question to investigate is whether the effectiveness of the EO depends on the accuracy of the beamlet dose calculations. Accordingly, the EO was applied to one of the head and neck cases, with the modification that the beamlets were computed on the patient phantom with water-equivalent CT values. This process is similar to a pencil-beam algorithm which neglects heterogeneity corrections.

### Results

Evaluation results are presented with DVH curves corresponding to the clinical plans produced by the TPS (labeled “Original”) and the plans produced by the enhanced optimization procedure (labeled “EO”). It is emphasized that all DVH's correspond to the final, deliverable plans, extracted from Eclipse. Some cases, particularly the brain and head-and-neck cases, involve many OAR's. For easier visualization, a DVH is only displayed if the OAR's objective penalty changed after the EO. Structures with no DVH's displayed may be assumed to have had their objectives met before and after the EO.

Enhanced Optimization with Original Objectives

Table 3 presents a numerical summary of the plans evaluated, showing the objective score before and after the EO process, along with the time for the greedy search to complete. Table 4 summarizes the changes to the doses corresponding to every objective which was not met in the Eclipse or EO plans.

TABLE 3
Summary of the enhanced optimization
results with original objectives.

| | Eclipse | EO | Score | Search |
|---|---|---|---|---|
| Plan | Score | Score | Improvement | Time (min) |
| Brain 1 | 98306 | 56918 | 42.1% | 28.2 |
| Brain 2 | 4920642 | 4600849 | 6.5% | 43.7 |
| Ped. Brain | 3109 | 1243 | 60.0% | 28.6 |
| H&N 1 | 398268 | 261458 | 34.4% | 91.5 |
| H&N 2 | 115757 | 108871 | 5.9% | 55.0 |
| Prostate 1 | 786884 | 426333 | 45.8% | 65.6 |
| Prostate 2 | 223 | 50 | 78.6% | 26.9 |

TABLE 4
Summary of the enhanced optimization results with original objectives.
Dose values are extracted directly from Eclipse.

| | OBJECTIVE | | ACTUAL DOSES | | Difference |
|---|---|---|---|---|---|
| OAR | Dose (Gy) | Volume % | Original (Gy) | EO (Gy) | (Gy) |

Brain 1

| | | | | | |
|---|---|---|---|---|---|
| Pituitary PRV | 45 | 0 | 49.28 | 46.41 | 2.87 |

Brain 2

| | | | | | |
|---|---|---|---|---|---|
| R. Cochlea PRV | 36.5 | 0 | 62.25 | 62.18 | 0.07 |
| Brainstem PRV | 54 | 0 | 57.75 | 57.59 | 0.16 |
| R. Lacrimal | 36 | 0 | 35.93 | 34.41 | 1.52 |
| R. Lacrimal PRV | 36 | 0 | 46.02 | 45.73 | 0.29 |
| Pituitary PRV | 45 | 0 | 53.25 | 51.94 | 1.31 |

Pediatric Brain

| | | | | | |
|---|---|---|---|---|---|
| L. Cochlea PRV | 35 | 0 | 39.35 | 38.49 | 0.86 |

Head and Neck 1

| | | | | | |
|---|---|---|---|---|---|
| Anterior Tongue | 74.5 | 0 | 76.11 | 75.32 | 0.79 |
| B. Stem PRV | 52.0 | 0 | 55.7 | 53.0 | 2.7 |
| R. Cochlea | 35 | 0 | 38.08 | 33.66 | 4.42 |
| L. Parotid | 59.5 | 0 | 62.69 | 61.11 | 1.58 |
| L. Parotid | 30.4 | 50.3 | 30.20 | 29.90 | 0.30 |

Head and Neck 2

| | | | | | |
|---|---|---|---|---|---|
| L. Cochlea | 30 | 0 | 29.29 | 26.63 | 2.66 |
| Ring 80 | 36 | 0 | 36.48 | 35.52 | 0.96 |
| Ring 50 | 21.5 | 0 | 21.51 | 21.17 | 0.34 |

Prostate 1

| | | | | | |
|---|---|---|---|---|---|
| Bladder | 53.4 | 0 | 54.5 | 54.25 | 0.25 |
| Penile Bulb | 53.4 | 0 | 53.82 | 53.68 | 0.14 |
| Rectum | 53.4 | 0 | 54.61 | 54.58 | 0.03 |

Prostate 2

| | | | | | |
|---|---|---|---|---|---|
| Rectum | 74 | 0 | 74.31 | 74.13 | 0.18 |

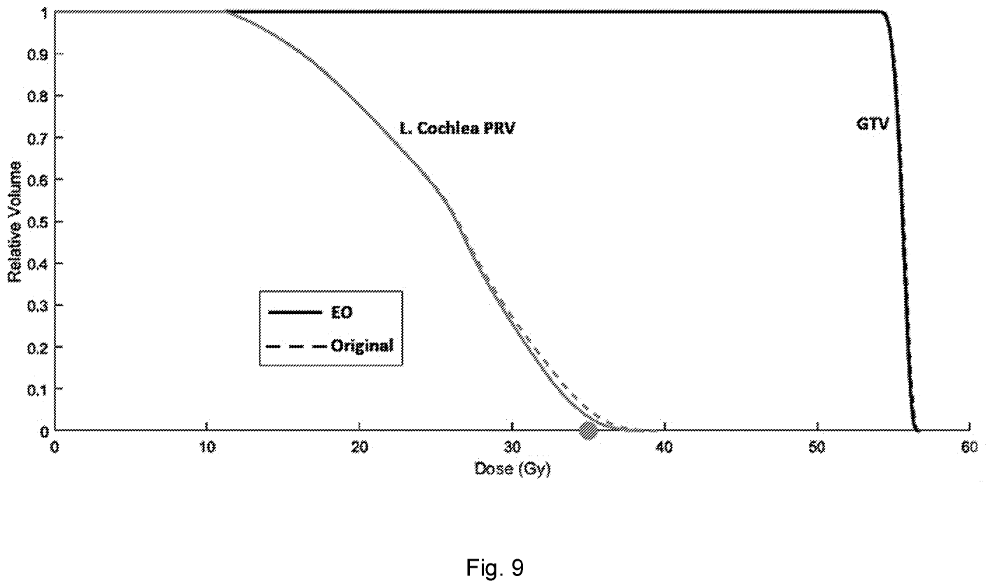

The first brain VMAT plan had non-overlapping PTV's (FIG. 7). The EO produced virtually no change to the 54 PTV, while slightly increasing the dose coverage to the 59.4 PTV. Improvement to the pituitary PRV DVH curve was apparent. Other structures saw some improvement to their DVH curves; however, their objectives were met prior to EO (i.e., they contributed no penalty score), and therefore are not shown in the DVH plot.

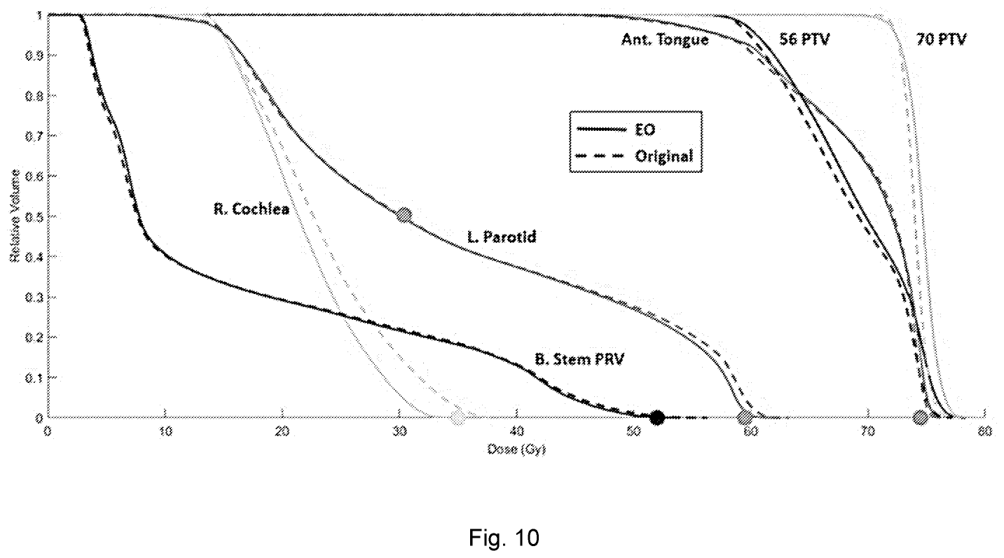

The second brain VMAT plan (FIG. 8) also saw a small decrease in dose homogeneity of the PTV while having noticeable improvement to the right cochlea PRV. Additionally, the right lacrimal and its PRV were improved. The pituitary also improved slightly, and the brainstem was virtually unchanged.

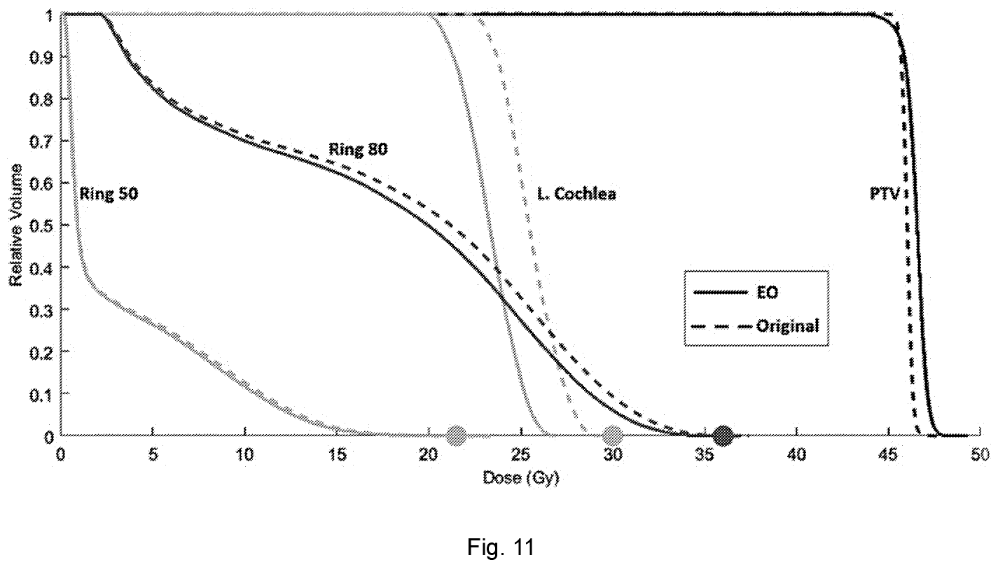

The pediatric brain VMAT plan (FIG. 9) had Eclipse objectives for the GTV instead of the PTV. There was a slight improvement to the left cochlear PRV, while the GTV was virtually unchanged. This plan was further investigated below.

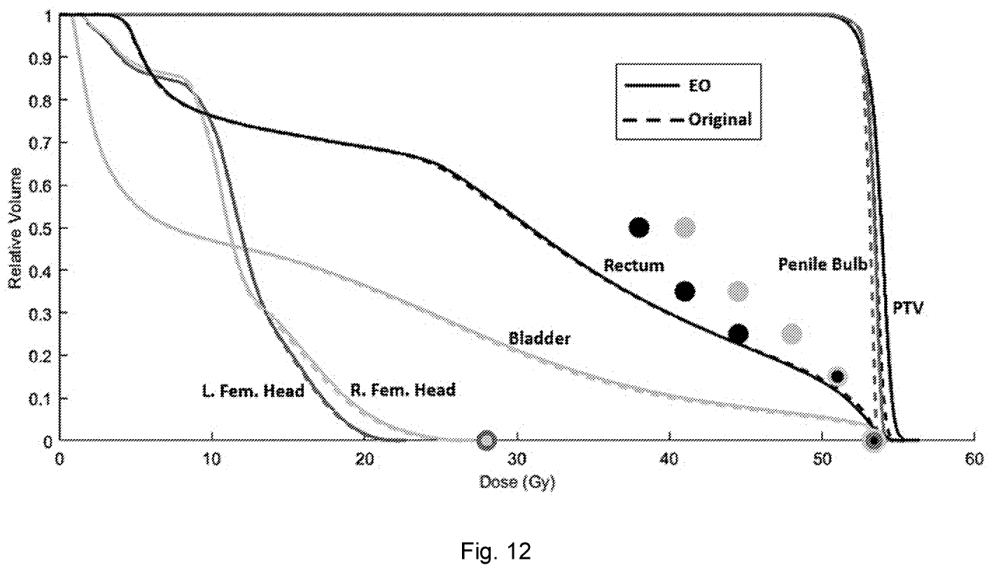

The first head and neck VMAT plan had a 70 PTV (to be boosted in a future plan) inside a 56 PTV (FIG. 10). There was a small improvement to the left parotid and larger improvements to the right cochlea. The 70 PTV was slightly less homogeneous.

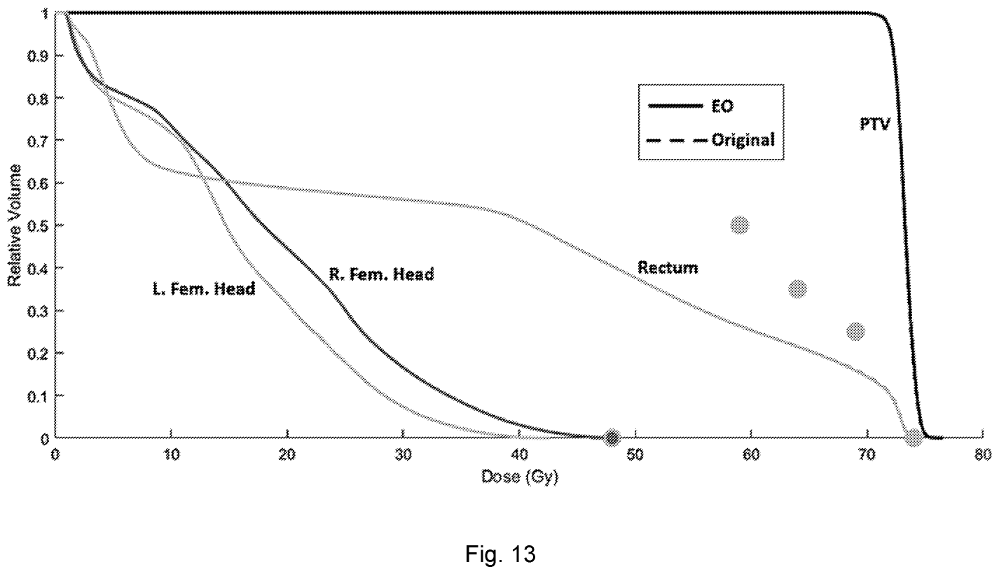

The second head and neck VMAT plan (FIG. 11) contained structures “Ring 50” and “Ring 80,” which were large trick planning structures created by the dosimetrist to optimize the 50% and 80% isodose regions, respectively. The Ring objectives were the only ones which were not met during TPS optimization. The EO reduced these penalties, resulting in a substantial improvement to the left cochlea. The changes to other structures were not noticeable.

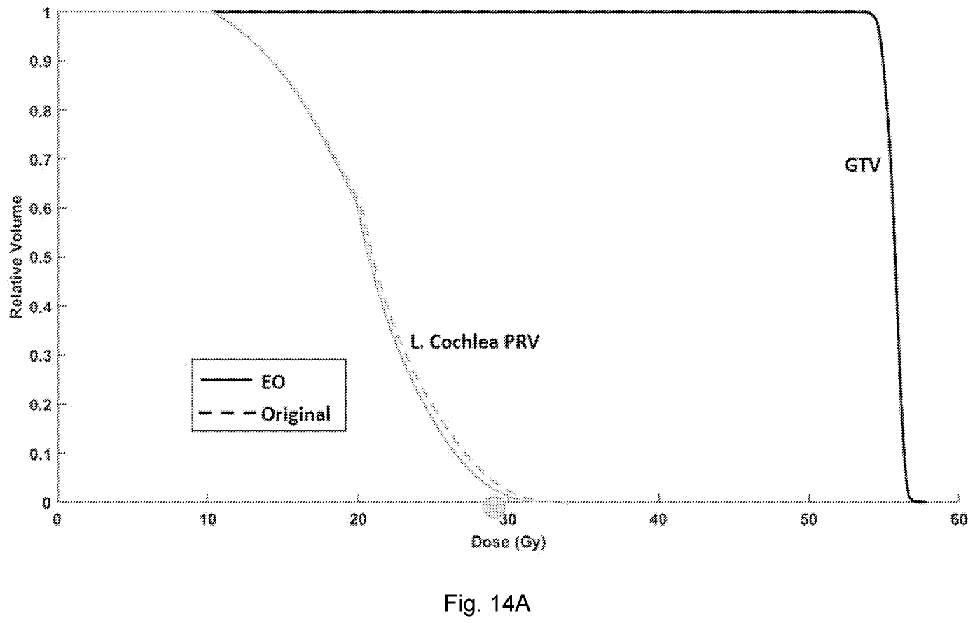

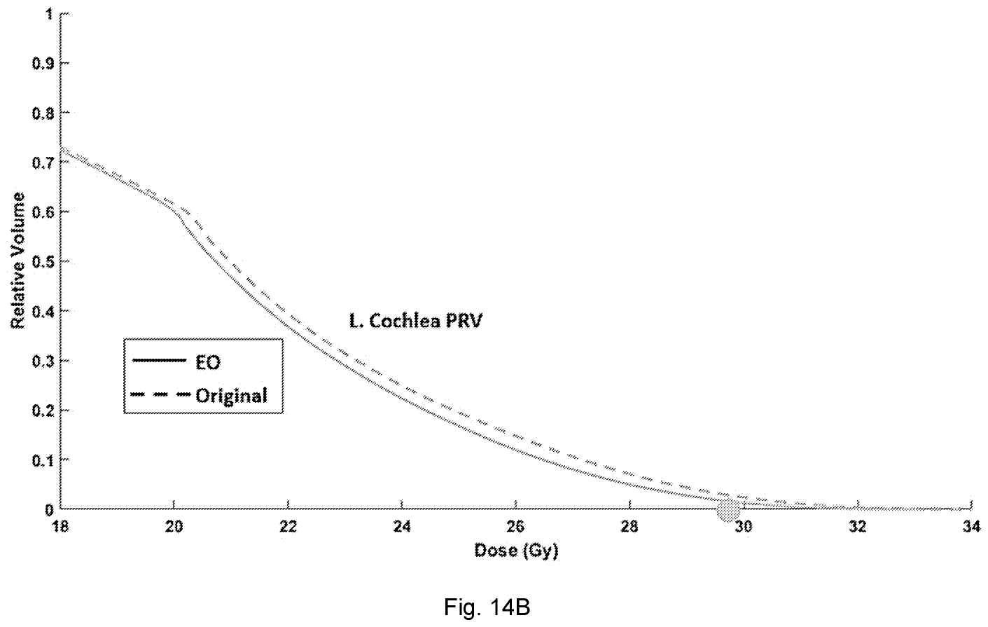

The two prostate cases are shown in (FIG. 12) and (FIG. 13). The DVH curves of both cases remained virtually unchanged by the EO process. This may be due to the Eclipse optimizer locating very stable minima for these relatively simple cases. The substantial reduction in objective scores are due to limitations in the Eclipse dose calculator (see Discussion).

Enhanced Optimization with Modified Objectives

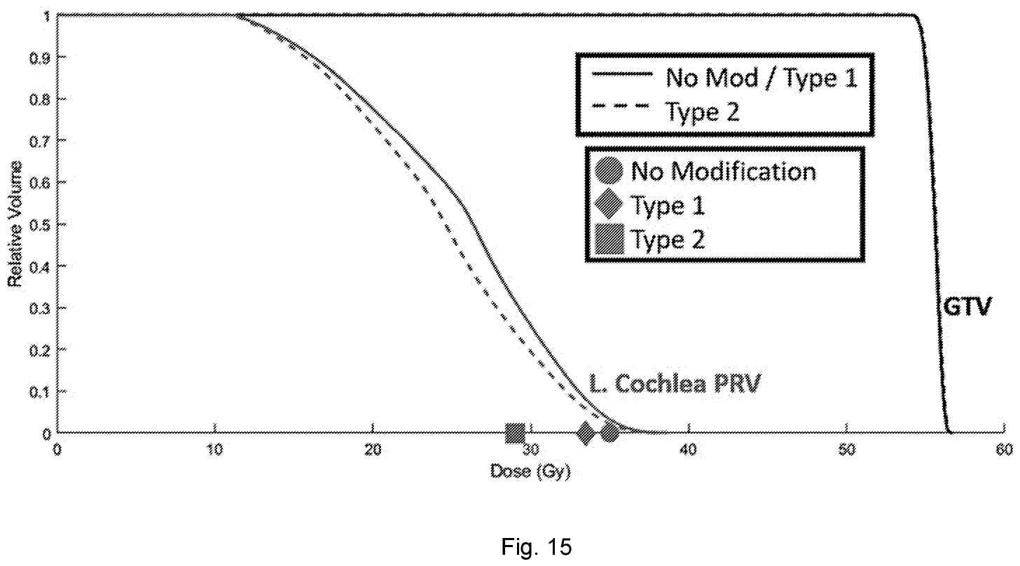

FIGS. 14A and 14B show the results of the Eclipse plan with the left cochlea PRV objective reduced to 29 Gy from 35 Gy. This reduced the maximum dose to 35.0 Gy from 39.5 Gy, while increasing the dose to some of the other OARs. The EO results are also shown, with a small further reduction to 34.0 Gy. Note that since every other objective was still met, the only contribution to the score was from this one objective.

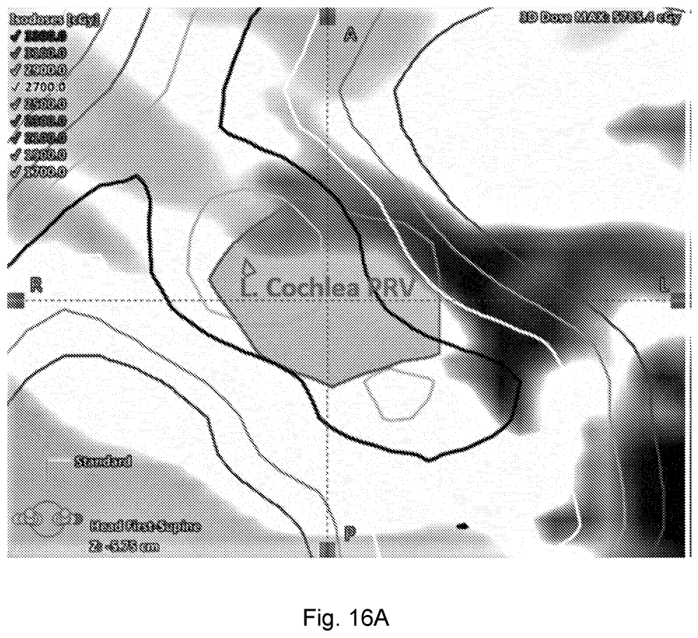

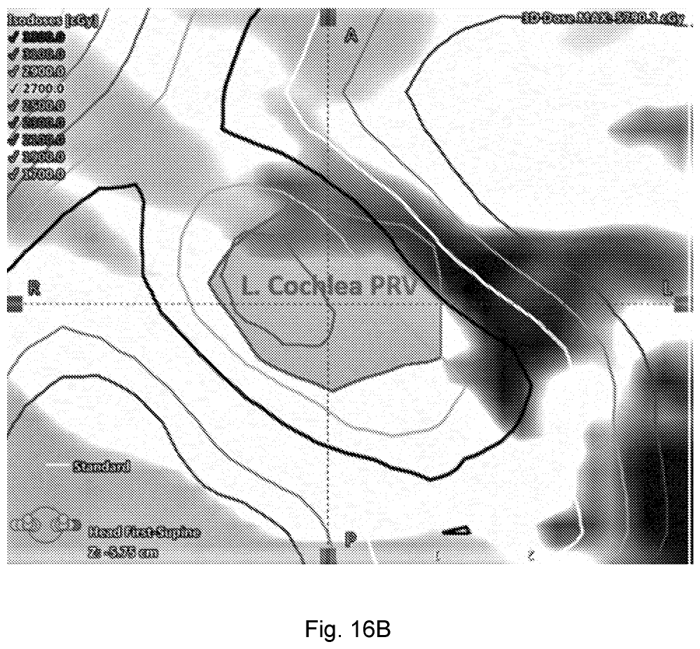

The EO was also applied to the original Eclipse plan (i.e., with an objective of 35 Gy), with the EO objective modified in two ways, as shown in Table 2, with the results shown in FIG. 15. The type 1 modification produced no change in the ternary optimization variables in Eq. (4). In other words, a reduction in maximum dose objective from 35 to 33.5 Gy for that single OAR did not produce a difference in penalty when the dose distributions were determined by the MC calculations, and therefore resulted in the same EO plan (see Discussion). The type 2 modification reduced the objective from 35 to 29 Gy, resulting in a noticeable improvement to the DVH. The isodose curves for these cases are shown in FIGS. 16A and 16B. The PTV DVH curve was virtually unchanged for each type of objective modification. Table 5 shows the plans which were created for this case: Eclipse, EO Unmodified, EO Modified (Type 1), and EO Modified (Type 2). The scores of each plan, where applicable, are indicated for the various objective functions: Original, Modified (Type 1), and Modified (Type 2).

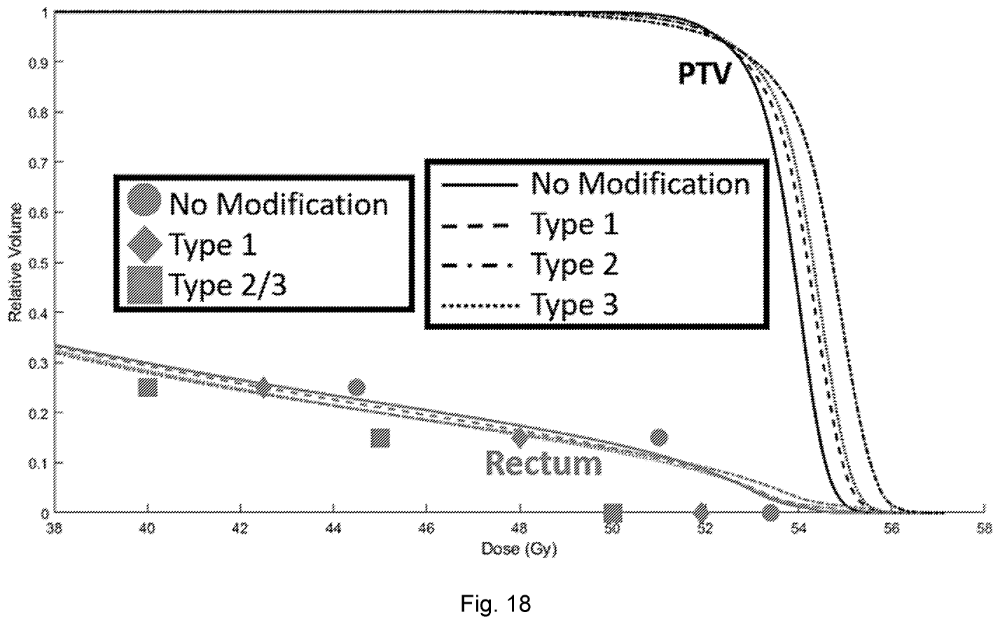

It should be noted that similar objective modifications were attempted for one of the prostate cases. They resulted in slight improvements to the rectum dose, as shown in FIG. 18, but substantially less homogeneous PTV dose (see Discussion).

### Differences in Eclipse and MC Calculations

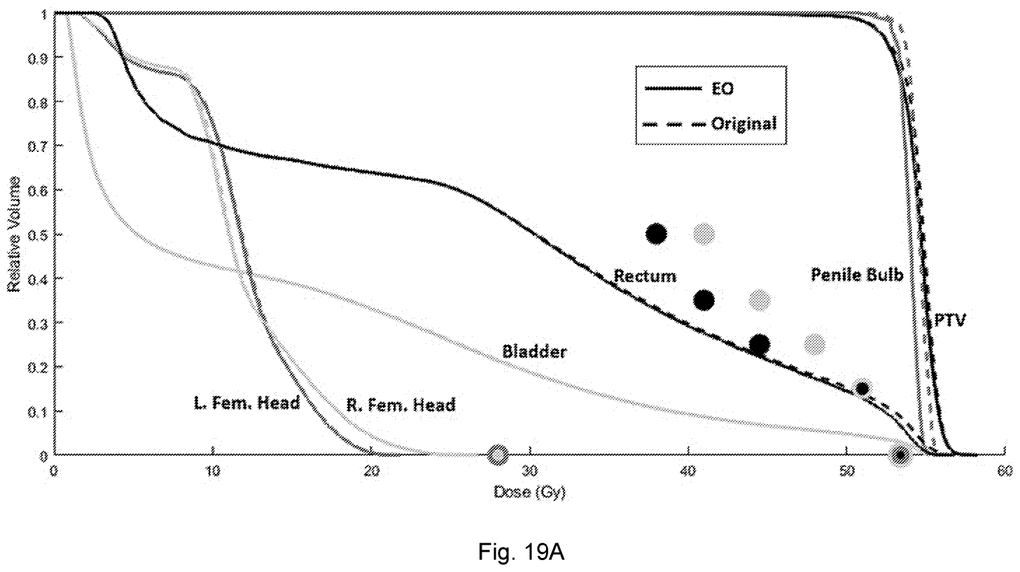

The EO process is based on doses calculated for VMAT plans using MC algorithms. In most cases, the plan dose values agree well with those calculated by the TPS. Some embodiments of the EO also provide for the calculation of DVH's for the VMAT plans, which also generally agree with the TPS DVHs. However, this study found some instances in which there were differences between the EO and Eclipse DVH's. That is, after the EO was applied to the plan, the new plan with the modified control points was imported into Eclipse. The dose was calculated, and the DVH data was exported for comparison with the EO DVH data. FIGS. 19A and 19B show a prostate plan comparison. Slight differences to the maximum doses to the rectum and penile bulb are evident. Since there are maximum-dose objectives to these OARs, the plan is optimized according to the EO, but not necessarily according to Eclipse. Therefore, the Eclipse plan may still be selected by the radiation oncologist, even though the EO plan is in fact of higher quality.

EO without Heterogeneity Corrections

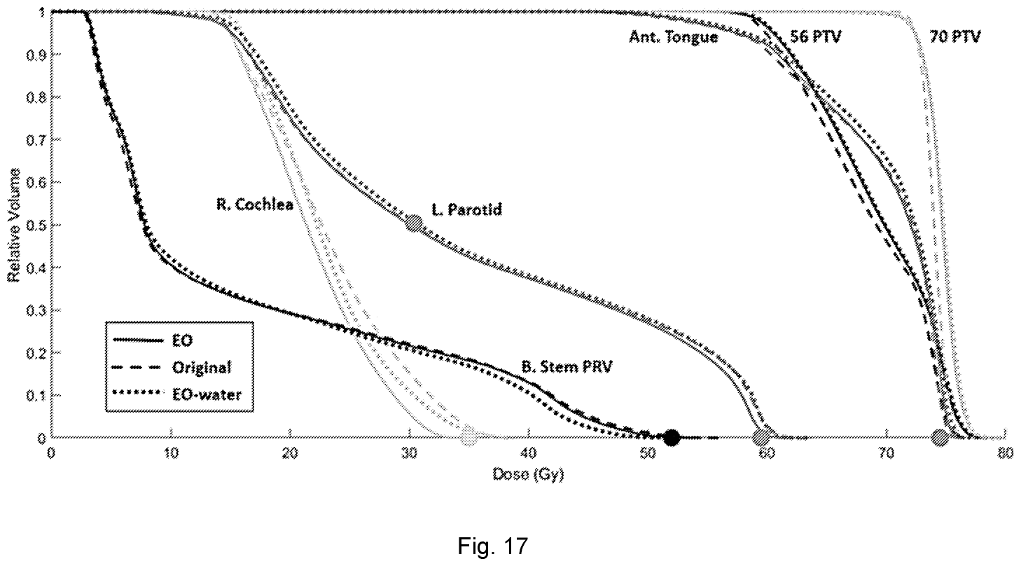

The process involving the beamlets calculated on a water-equivalent CT was termed the EO-water. FIG. 17 shows a comparison of the Eclipse, EO, and EO-water plans for a head and neck case. The EO-water provides some improvement to the Eclipse plan, but was not as effective as the EO. This can also be seen by the objective function values, shown in Table 6. These results illustrate the benefit of employing a full MC calculation engine to obtain the beamlet dose matrices.

TABLE 5
Objective function scores for pediatric brain plans. The
EO plans correspond to different objectives on one OAR.
Unmodified and Type 1 resulted in the same plan. Type
2 DVH was only different in high-dose region (see text).
All EO plans had lower scores than the Eclipse plans.

| | Original | Modified | Modified |
|---|---|---|---|
| Plan | Score | Type 1 Score | Type 2Score |
| Eclipse | 3109 | 24415 | 92495 |
| EO Unmodified | 1243 | 20137 | 72213 |
| EO Mod. Type 1 | 1243 | 20137 | 72213 |
| EO Mod. Type 2 | 1243 | 20137 | 69746 |

TABLE 6
Comparison of objective score and dose differences between the Eclipse,
EO, and EO-water plans for each OAR objective of a head and neck
case. The EO score was lowest for every objective except one.

| | OBJECTIVE | | ACTUAL DOSE | | | OBJECTIVE SCORES | | |
|---|---|---|---|---|---|---|---|---|
| OAR | Dose | Volume | Eclipse | EO | EO-water | Eclipse | EO | EO-water |
| Ant. Tongue | 74.5 Gy | 0.0% | 76.1 Gy | 76.1 Gy | 76.8 Gy | 51313 | 38056 | 79898 |
| B. Stem PRV | 52.0 Gy | 0.0% | 55.7 Gy | 53.0 Gy | 51.5 Gy | 549823 | 318296 | 315684 |
| R. Cochlea | 35.0 Gy | 0.0% | 38.1 Gy | 34.0 Gy | 38.5 Gy | 2948 | 57 | 340 |
| L. Parotid | 59.5 Gy | 0.0% | 62.7 Gy | 61.7 Gy | 63.0 Gy | 11324 | 9059 | 16125 |
| L. Parotid | 30.4 Gy | 50.3% | 30.2 Gy | 29.9 Gy | 30.6 Gy | 0 | 0 | 115 |
| | | | | | TOTALS: | 615418 | 365468 | 412162 |

### Discussion

In every evaluation performed, the enhanced optimization process was able to achieve a more optimal dose distribution, as indicated by the scores in Table 3. This was particularly true for the complex cases (e.g., brain and head and neck), whose Eclipse plans did not meet one or more of their objectives. However, an EO score improvement does not provide a complete picture of the plan quality. Both prostate case scores were substantially improved (by 46% and 79%), and yet their DVH's remained virtually unchanged, when Eclipse was used to calculate the EO DVH's. This is the result of differences in dose calculations between the Eclipse and MC algorithms, as explained above.

From an optimization standpoint, the prostate cases are relatively simple. The targets are approximately spherical and are centrally located. The adjacent OAR's (bladder and rectum) are relatively large structures. This offers more flexibility to the optimizer in terms of potential locations within the OAR in which to spread dose. It also results in the OAR DVH being less volatile when subject to small changes to the dose distribution. The small dose perturbations used by the EO process will not substantially change the OAR DVH's. In contrast, brain and head and neck cases involve irregularly-shaped targets, which may be adjacent to small OAR's. Small changes to the dose may result in large changes to the OAR DVH's, and therefore to the plan score. Therefore, the EO may offer a greater benefit to complex plans, because the dose perturbations permit the system to explore the neighboring optimization space for improvements.

It is also apparent that relative raw score reduction had less impact on plan enhancement than absolute score reduction. For example, the score for the pediatric brain case was reduced by 60% (1866 points), whereas the second brain case was reduced by 6.5% (31215 points). However, the DVH's for these cases indicate that the second brain case benefited more from EO. This is also true in the second prostate case, whose absolute score reduction of 183 points (the lowest of the 7 cases), translated into a relative reduction of 78.6%, (the highest of the cases).

EO has great clinical potential, especially for plans with an OAR which may be of particular concern. For example, a radiation oncologist in our center indicated that sparing of the cochlear structures in a pediatric brain patient is critical to a lifetime of preserved hearing function. This provided the motivation to apply the modified objective EO process to this particular structure. When such a case is identified, the planning may be performed as usual, and then the EO can be run overnight, for comparison with the original plan the next day. The process may be greatly streamlined and automated through the use of scripting.

The VMAT plans evaluated for this study all involved 6-MV energies and a Varian Trilogy linac. However, we have since employed the BEAMnrc module to model a TrueBeam linac with a high-definition MLC, and incorporated phase space files to support MC calculations with higher energies and flattening-filter free treatment modes. These new calculation capabilities will permit the investigation of EO applied to SBRT VMAT plans, in which DVH planning objectives are critical.

The beamlet calculation time depends linearly on the number of beamlets. Therefore, a high-definition MLC plan would use approximately double the calculation time (assuming the targets are within the high-definition region). The beamlet calculation time, or number of particles required to achieve the same level of uncertainty, scales as the inverse of voxel size (along one dimension). Therefore, the time is proportional to the number of voxels in the calculation volume.

The beamlet calculation is the first step of the EO, but it is only required once, before the optimization is performed. Currently, the maximum time to compute beamlets is approximately 18 hours for one plan. However, this includes time spent in the computer cluster's batch queue. A dedicated cluster and efficient parallelization of jobs would reduce this time, as would ever-increasing processor power. Once the beamlet matrices are calculated, the greedy search ran only 30-90 minutes, as shown in Table 3. The greedy search was selected due to its simplicity; however, any discrete optimization algorithm which can be applied to ternary variables may be used. Genetic algorithms and other methods are currently being investigated. In addition, the order in which the ternary variables are modified is arbitrary. In our implementation, the search begins with the first leaf of the first control point, and proceeds sequentially. However, any leaf may serve as a starting point, and the order of the search may be altered. This may potentially affect the solution, since the effect of one perturbation on the objective function depends on which other perturbations have already been applied.

The beamlets act as perturbations to the original TPS-optimized plan. The width of the beamlets in the isocenter plane is determined by the width of the central leaves of the MLC, which was 0.5 cm in this study. This may be modified for other MLC types (e.g., 0.25 cm for high-definition MLC's). The length of the beamlets (i.e., in the direction of leaf travel) was selected to be 0.5 cm, although this value may be adjusted higher or lower based on the plan type. For example, as discussed above, prostate plans are less sensitive to perturbations to the dose distribution, and may respond to larger beamlets in the EO process. Plans involving very small target structures and OAR's, such as those used for stereotactic body radiation therapy (SBRT) and stereotactic radiosurgery (SRS) cases, may benefit from smaller beamlets. A further embodiment utilizes variable beamlet sizes, in which various-length beamlets are calculated (e.g., 0.2 cm to 1.0 cm, in steps of 0.2 cm). This will increase the number of beamlets and their calculation time, and change the EO variable type from ternary to a larger discrete type.

As shown in this study, DVH objectives may be modified from their original values. This has the effect of changing the objective function, and therefore the optimization space. However, when the original TPS plan is used as the starting point, and the EO objectives are modified, only a new greedy search is required, not a recalculation of the beamlet matrices; various combinations of objectives and weightings may be investigated in this way. The modified pediatric brain cochlear objective did reduce the dose to the left cochlea. However, the prostate plan rectum objectives could not be reduced without incurring a substantial reduction to the homogeneity of the PTV dose. This demonstrates that the EO may open up a new area of treatment planning, in which a planner may acquire skill and experience in both the TPS and enhanced optimization processes.

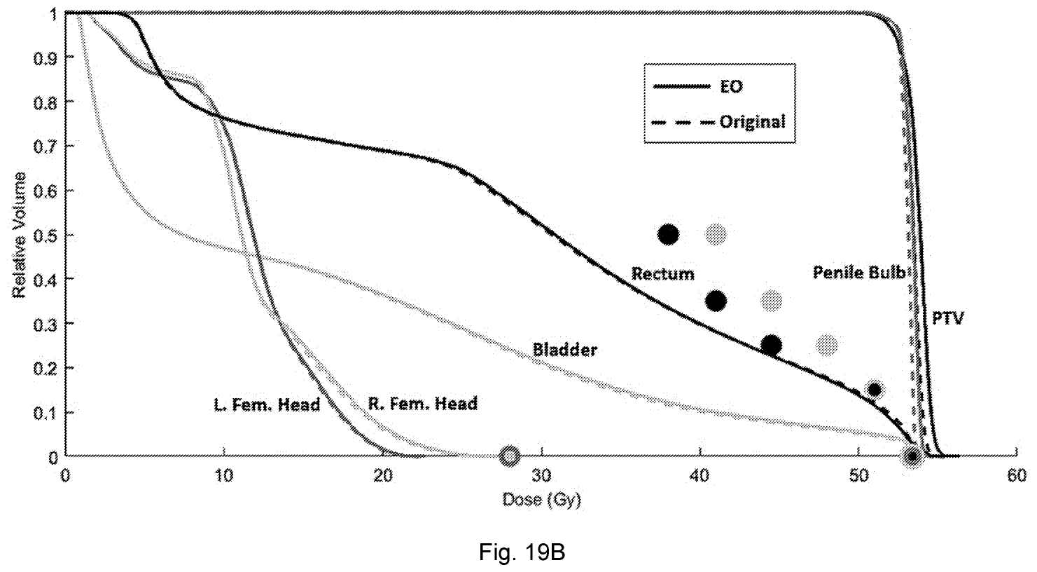

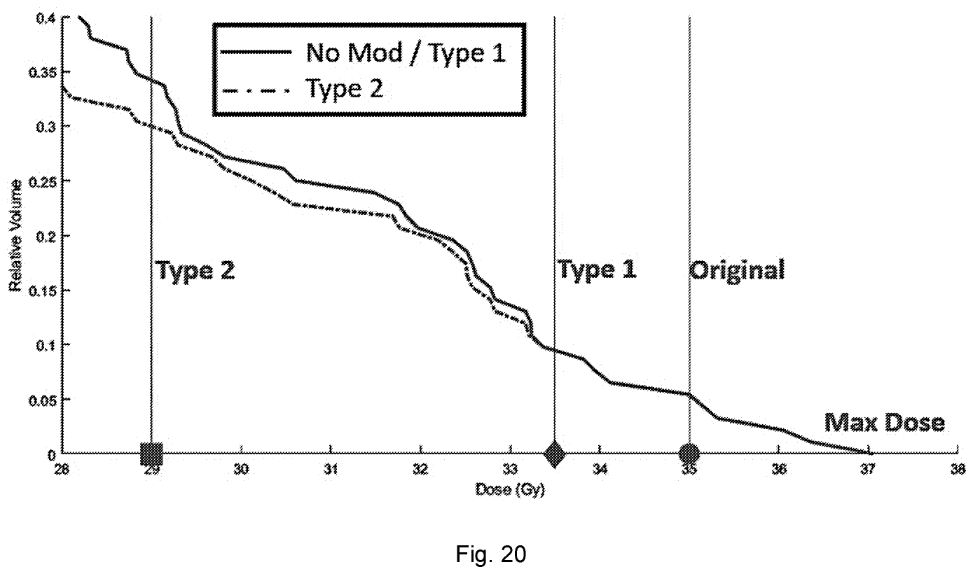

Another issue that was encountered involved running the EO with the modified objectives for the pediatric brain case. As shown in FIG. 20, the Unmodified (35 Gy), Type 1 (33.5 Gy), and Type 2 (29 Gy) maximum-dose objectives were investigated for the left cochlea PRV. The Unmodified and Type 1 objectives produced identical plans, as indicated by the DVH's. This was also verified by examining the values of x in Eq. (4). However, using the Type 2 objective resulted in a reduction in dose to the OAR. This plan was not found during the Type 1 optimization, because it produces the same objective penalty as that of the Unmodified plan. Both Type 2 and Unmodified DVH's coincide at doses above about 33 Gy. This is the reason Table 5 has identical values for the EO Unmodified and EO Modified Type 1 plans. Therefore, a sufficient reduction in the objective is required to produce a change in the plan.

We have shown that EO may improve VMAT plan quality, particularly with respect to OAR objectives. However, there are several aspects that should be considered. It requires the calculation of several thousand beamlets for each VMAT arc. Although high-performance computing hardware can be applied to this problem, many radiation oncology centers do not readily have access to such resources. The EO also does not provide significant improvements in some cases that were investigated, particularly prostate plans. And in its current workflow implementation, it would place an additional time burden on the treatment planner, who first completes a standard VMAT planning/approval cycle with the MD involving the original plan, and then a comparison/approval step involving the EO plan.

There were certain limitations to this study. It involved a small (N=7) number of plans, covering three anatomic sites. It was apparent that the EO results were site dependent, but more work is required to determine what anatomy/objective combinations have the potential for significant EO improvements. The EGSnrc platform could be replaced with any computational framework which supports Monte Carlo beamlet calculation scripts. This approach may reduce the overall calculation time required by the EO.

### Weight/Intensity-Level Linear Optimization

In another aspect, the present disclosure provides another VMAT Treatment Plan optimization method called the Weight/Intensity-Level Linear Optimization (WILLOw) method. In this method, weights, or intensities, of each CP are used as variables. The starting value of each weight is given by the TPS (e.g., Eclipse) as the meterset. By varying each meterset, we can increase or decrease the contribution from each CP. This requires the CP dose matrices to be calculated independently of the TPS. To accomplish this, the monte carlo (MC) dose calculation routine is used (e.g., EGSnrc). MC algorithms are considered the gold standard in accuracy for calculating dose distributions, with the drawback of slow run times. By employing a supercomputer cluster to parallelize the computations, we were able to perform the WILLOw method calculations within a few hours per VMAT plan.

The EO approach described above improves VMAT plans by varying the shape of each CP aperture. Embodiments of the WILLOw approach vary the weights of each CP. This provides the advantage of making each variable continuous, rather than discrete, and therefore amenable to any continuous-variable optimization algorithm. In addition, the dose matrix for each CP is only required to be calculated once, since varying the weight is equivalent to multiplying the matrix by a single scale factor (a separate factor for each CP). As the weights are varied, the new dose is calculated by summing the individual CP dose matrices. The objective function is evaluated, and optimization proceeds iteratively until a (possibly local) minimum is located.

Once a new optimal set of CP weights is determined, the VMAT plan is updated and can be imported into Eclipse as part of a DICOM file. This modified plan is called the WILLOw plan. A full calculation with the WILLOw plan may then be performed in Eclipse, in order to ensure that the plan is clinically deliverable, and to display the modified dose distribution (e.g., DVHs, isodose curves, etc.) This allows the radiation oncologist to compare the WILLOw plan with the original. In addition, if the WILLOw plan is selected as the treatment plan, it can be used in regular clinical workflow (e.g., QA measurements and transfer to the linac) without any further modification.

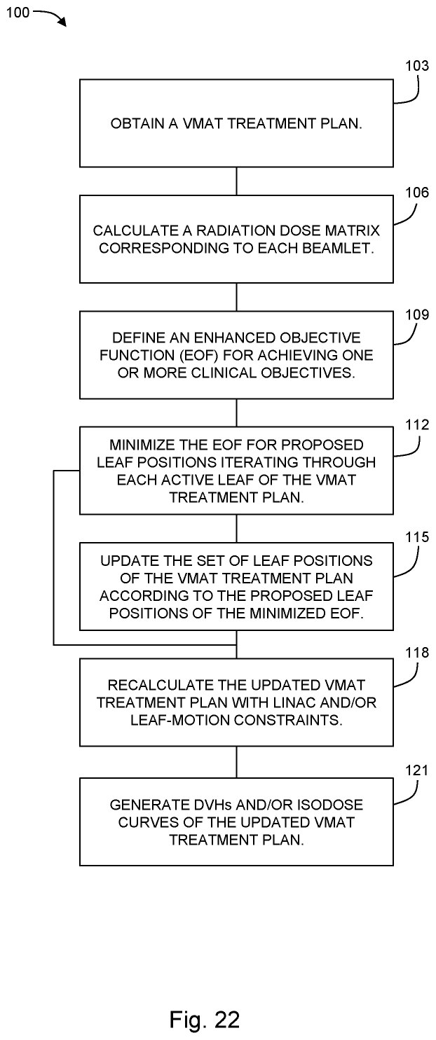

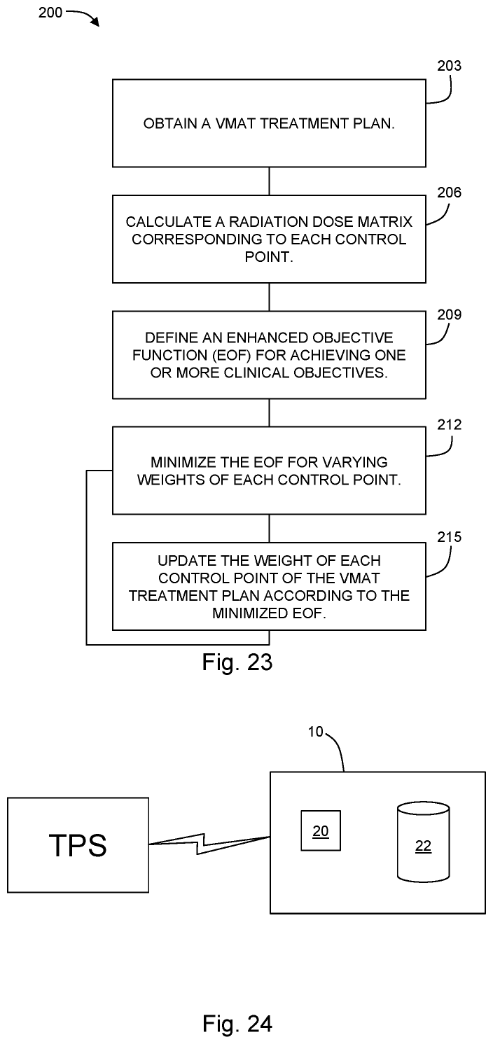

With respect to FIG. 23, the present disclosure may be embodied as a method 200 for optimizing a VMAT treatment plan. The method includes obtaining 203 a VMAT treatment plan. For example, the VMAT treatment plan may be obtained from a TPS. The VMTA treatment plan includes a plurality of control points, each control point having a weight corresponding to an intensity of the linac beam at the associated control point. It should be noted that “intensity” as used herein may refer to an intensity of the linac beam and/or the time at which the linac beam remains at or near a position. As such, the maximum “intensity” need not be limited by the maximum beam power of the linac, but may be further increased by allowing the linac beam to slow down and/or stop at a given position.

An EOF is defined 209 for achieving one or more clinical objectives. The EOF may include clinical objectives to achieve at least a minimum dose to a target volume and minimize a dose to an organ at risk. There may be more than one target volumes and/or more than one OARs included within the scope of an EOF within the present scope. The EOF may be the same as the objective function used to generate the original VMAT treatment plan (e.g., generated by the TPS). In some embodiments, the EOF is different from the objective function used to generate the original VMAT treatment plan.

A radiation dose matrix is calculated 206 for each control point. The radiation control matrices may be calculated using MC dose calculation (MC routines). The EOF is minimized 212 iterating through a varying weight of each control point, which corresponds to increasing or decreasing (from the original weight) the associated radiation dose matrix by a scale factor. The minimization may be performed using a continuous optimization routine. For example, the minimization may use an unbounded continuous optimization, a bounded continuous optimization, or other optimization. In some embodiments, the weight is varied from 0 (e.g., no beam energy at the control point—beam off, MLC leaves closed, or otherwise) to a predetermined maximum weight.

The VMAT treatment plan is updated 215 with the weight of each control point in accordance with the minimized EOF.

In another aspect, the present disclosure may be embodied as a system for performing any of the methods described herein. For example, a system 10 may include a processor 20 and a memory 22 in electronic communication with the processor. The memory may comprise instructions for the processor to perform an embodiment of method 100 described above—i.e., obtain a VMAT treatment plan from a treatment planning system (TPS), the VMAT treatment plan having a plurality of control points, each control point having a set of leaf positions corresponding a set of leaves of a multileaf collimator (MLC) in a field of a linear accelerator (linac); calculate a radiation dose matrix corresponding to each beamlet, wherein a beamlet is the change in field when an MLC leaf is moved a predetermined unit distance; define an enhanced objective function (EOF) for achieving one or more clinical objectives, including a achieving at least a minimum dose to a target volume and minimizing a dose to an organ at risk; minimize the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix; and update the set of leaf positions of the VMAT treatment plan according to the proposed leaf positions of the minimized EOF.

In another embodiment, the memory may comprise instructions for the processor to perform an embodiment of method 200 described above—i.e., obtain an optimized VMAT treatment plan from a treatment planning system (TPS), the VMAT treatment plan having a plurality of control points, each control point having a weight corresponding to an intensity of the linear accelerator (linac) beam at the associated control point; define an enhanced objective function (EOF) for achieving one or more clinical objectives, including achieving at least a minimum dose to a target volume and minimizing a dose to an organ at risk; calculate a radiation dose matrix associated with each control point; minimize the EOF iterating through a varying weight of each control point, which corresponds to increasing or decreasing of the associated dose matrix by a scale factor; and update the weight of each control point of the VMAT treatment plan according to the minimized EOF.

In another aspect, the present disclosure may be embodied as a non-transitory computer-readable medium encoded with computer-executable instructions, which when executed by a processor cause the processor to perform any of the methods described herein (such as, for example, embodiments of method 100 or method 200).

The term processor is intended to be interpreted broadly. For example, in some embodiments, the processor includes one or more modules and/or components. Each module/component executed by the processor can be any combination of hardware-based module/component (e.g., graphics processing unit (GPU), a field-programmable gate array (FPGA), an application specific integrated circuit (ASIC), a digital signal processor (DSP)), software-based module (e.g., a module of computer code stored in the memory and/or in the database, and/or executed at the processor), and/or a combination of hardware- and software-based modules. Each module/component executed by the processor is capable of performing one or more specific functions/operations as described herein. In some instances, the modules/components included and executed in the processor can be, for example, a process, application, virtual machine, and/or some other hardware or software module/component. The processor can be any suitable processor configured to run and/or execute those modules/components. The processor can be any suitable processing device configured to run and/or execute a set of instructions or code. For example, the processor can be a general purpose processor, a central processing unit (CPU), an accelerated processing unit (APU), a field-programmable gate array (FPGA), an application specific integrated circuit (ASIC), a digital signal processor (DSP), graphics processing unit (GPU), microprocessor, controller, microcontroller, and/or the like. In a particular example, the processor is a supercomputer cluster of processors, GPUs, and/or other components, such as the supercomputer resources of the Center for Computational Research at the University at Buffalo.

Further embodiments are provided in the examples below.

Example 1. A method for optimizing a volumetric modulated arc therapy (VMAT) treatment plan, comprising: obtaining a VMAT treatment plan from a treatment planning system (TPS), the VMAT treatment plan having a plurality of control points, each control point having a set of leaf positions corresponding to a set of leaves of a multileaf collimator (MLC) in a field of a linear accelerator (linac); calculating a radiation dose matrix corresponding to each beamlet, wherein a beamlet is the change in field when an MLC leaf is moved a predetermined unit distance; defining an enhanced objective function (EOF) for achieving one or more clinical objectives, including achieving at least a minimum dose to a target volume and minimizing a dose to an organ at risk; minimizing the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix; and updating the set of leaf positions of the VMAT treatment plan according to the proposed leaf positions of the minimized EOF.

Example 2. The method of example 1, wherein the minimizing and updating steps are performed for each control point of the VMAT treatment plan.

Example 3. The method of any one of examples 1 or 2, wherein the one or more clinical objectives of the EOF are different from clinical objectives used to generate the VMAT treatment plan.

Example 4. The method of any one of example 1-3, wherein the beamlet dose matrices are calculated using Monte Carlo routines.

Example 5. The method of any one of example 1-4, wherein the proposed leaf position of each leaf is represented by a vector (x) of ternary leaf variables, and the EOF (ƒ_{E}) is a function of the vector (ƒ_{E}(x)).

Example 6. The method of example 5, wherein x=[x₁, x₂, . . . , xₙ], where n is the number of active leaves in the VMAT treatment plan and xᵢ∈{−1, 0, 1}, where i is an index value, −1 is a move of 1 unit distance into the field, 1 is a move of 1 unit distance out of the field, and 0 is an unchanged leaf position.

Example 7. The method of example 6, wherein the EOF is ƒ_{E}(x)=ΣⱼΣᵢWⱼ[(d_{j,min}−dᵢ(x))²*H{d_{j,min}−dᵢ(x)}+(dᵢ(x)−d_{j,max})²*H{dᵢ(x)−d_{j,max}}], where j is an index of clinical objectives, d_{j,min} is a minimum-dose objective, d_{j,max} is a maximum-dose objective, i is a voxel, Wⱼ is a weight for each clinical objective, and H is either 1 or 0 to eliminate terms which do not violate the clinical objective.

Example 8. The method of any one of example 1-7, further comprising recalculating the updated VMAT treatment plan with linac and/or leaf-motion constraints.

Example 9. The method of example 8, further comprising generating dose-volume histograms and/or isodose curves of the updated VMAT treatment plan.

Example 10. A VMAT treatment plan optimization system, comprising: a processor; and a memory in electronic communication with the processor, the memory comprising instructions for the processor to: obtain a VMAT treatment plan from a treatment planning system (TPS), the VMAT treatment plan having a plurality of control points, each control point having a set of leaf positions corresponding a set of leaves of a multileaf collimator (MLC) in a field of a linear accelerator (linac); calculate a radiation dose matrix corresponding to each beamlet, wherein a beamlet is the change in field when an MLC leaf is moved a predetermined unit distance; define an enhanced objective function (EOF) for achieving one or more clinical objectives, including achieving at least a minimum dose to a target volume and minimizing a dose to an organ at risk; minimize the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix; and update the set of leaf positions of the VMAT treatment plan according to the proposed leaf positions of the minimized EOF.

Example 11. The system of example 10, wherein the processor performs the minimizing and updating steps for each control point of the VMAT treatment plan.

Example 12. The system of any one of example 10 or 11, wherein the one or more clinical objectives of the EOF are different from clinical objectives used to generate the VMAT treatment plan.

Example 13. The system of any one of examples 10-12, wherein the processor calculates the beamlet dose matrices using Monte Carlo routines.

Example 14. The system any one of examples 10-13, wherein the proposed leaf position of each leaf is represented by a vector (x) of ternary leaf variables, and the EOF (ƒ_{E}) is a function of the vector (ƒ_{E}(x)).

Example 15. The system of example 14, wherein x=[x₁, x₂, . . . , xₙ], where n is the number of active leaves in the VMAT treatment plan and xᵢ∈{−1, 0, 1}, where i is an index value, −1 is a move of 1 unit distance into the field, 1 is a move of 1 unit distance out of the field, and 0 is an unchanged leaf position.

Example 16. The system of example 15, wherein the EOF is ƒ_{E}(x)=ΣⱼΣᵢWⱼ[(d_{j,min}−dᵢ(x))²*H{d_{j,min}−dᵢ(x)}+(dᵢ(x)−d_{j,max})²*H{dᵢ(x)—d_{j,max}}], where j is an index of clinical objectives, d_{j,min} is a minimum-dose objective, d_{j,max} is a maximum-dose objective, i is a voxel, Wⱼ is a weight for each clinical objective, and H is either 1 or 0 to eliminate terms which do not violate the clinical objective.

Example 17. The system of any one of examples 10-16, wherein the processor is further instructed to recalculate the updated VMAT treatment plan with linac and/or leaf-motion constraints.

Example 18. The system of example 17, wherein the processor is further instructed to generate dose-volume histograms and/or isodose curves of the updated VMAT treatment plan.

Example 19. A non-transitory computer-readable medium encoded with computer-executable instructions, which when executed by a processor, cause the processor to: obtain a VMAT treatment plan from a treatment planning system (TPS), the VMAT treatment plan having a plurality of control points, each control point having a set of leaf positions corresponding a set of leaves of a multileaf collimator (MLC) in a field of a linear accelerator (linac); calculate a radiation dose matrix corresponding to each beamlet, wherein a beamlet is the change in field when an MLC leaf is moved a predetermined unit distance; define an enhanced objective function (EOF) for achieving one or more clinical objectives, including achieving at least a minimum dose to a target volume and minimizing a dose to an organ at risk; minimize the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix; and update the set of leaf positions of the VMAT treatment plan according to the proposed leaf positions of the minimized EOF.

Example 20. A method for optimizing a volumetric modulated arc therapy (VMAT) treatment plan, comprising: obtaining a VMAT treatment plan from a treatment planning system (TPS), the VMAT treatment plan having a plurality of control points, each control point having a weight corresponding to an intensity of the linear accelerator (linac) beam at the associated control point; defining an enhanced objective function (EOF) for achieving one or more clinical objectives, including achieving at least a minimum dose to a target volume and minimizing a dose to an organ at risk; calculating a radiation dose matrix associated with each control point; minimizing the EOF iterating through a varying weight of each control point, which corresponds to increasing or decreasing the associated radiation dose matrix by a scale factor; and updating the weight of each control point of the VMAT treatment plan according to the minimized EOF.

Example 21. The method of example 20, wherein the dose matrix for each control point is calculated using a monte carlo (MC) dose calculation.

Example 22. The method of any one of examples 20 or 21, wherein the one or more clinical objectives of the EOF are different from clinical objectives used to generate the VMAT treatment plan.

Example 23. The method of any one of examples 20-22, wherein the minimization step is performed using a continuous optimization routine.

Example 24. The method of any one of examples 20-22, wherein the weight is varied from 0 to a predetermined maximum weight.

Example 25. A VMAT treatment plan optimization system, comprising: a processor; and a memory in electronic communication with the processor, the memory comprising instructions for the processor to: obtain an optimized VMAT treatment plan from a treatment planning system (TPS), the VMAT treatment plan having a plurality of control points, each control point having a weight corresponding to an intensity of the linear accelerator (linac) beam at the associated control point; define an enhanced objective function (EOF) for achieving one or more clinical objectives, including achieving at least a minimum dose to a target volume and minimizing a dose to an organ at risk; calculate a radiation dose matrix associated with each control point; minimize the EOF iterating through a varying weight of each control point, which corresponds to increasing or decreasing of the associated dose matrix by a scale factor; and update the weight of each control point of the VMAT treatment plan according to the minimized EOF.

Example 26. The system of example 25, wherein the dose matrix for each control point is calculated using a monte carlo (MC) dose calculation.

Example 27. The system of any one of examples 25 or 26, wherein the one or more clinical objectives of the EOF are different from clinical objectives used to generate the VMAT treatment plan.

Example 28. The system of any one of examples 25-27, wherein the minimization step is performed using a continuous optimization routine.

Example 29. The system of any one of examples 25-28, wherein the weight is varied from 0 to a predetermined maximum weight.

Example 30. A non-transitory computer-readable medium encoded with computer-executable instructions, which when executed by a processor, cause the processor to: obtain an optimized VMAT treatment plan from a treatment planning system (TPS), the VMAT treatment plan having a plurality of control points, each control point having a weight corresponding to an intensity of the linear accelerator (linac) beam at the associated control point; define an enhanced objective function (EOF) for achieving one or more clinical objectives, including achieving at least a minimum dose to a target volume and minimizing dose to one or more organs at risk; calculate a dose matrix associated with each control point; minimize the EOF iterating through a varying weight of each control point, which corresponds to increasing or decreasing of the associated dose matrix by a scale factor; and update the weight of each control point of the VMAT treatment plan according to the minimized EOF.

Although the present disclosure has been described with respect to one or more particular embodiments, it will be understood that other embodiments of the present disclosure may be made without departing from the spirit and scope of the present disclosure.

What is claimed is:

1. A method for optimizing a volumetric modulated arc therapy (VMAT) treatment plan, comprising:
obtaining a VMAT treatment plan from a treatment planning system (TPS), the VMAT treatment plan having a plurality of control points, each control point having a set of leaf positions corresponding to a set of leaves of a multileaf collimator (MLC) in a field of a linear accelerator (linac);
calculating a radiation dose matrix corresponding to each beamlet, wherein a beamlet is the change in field when an MLC leaf is moved a predetermined unit distance;
defining an enhanced objective function (EOF) for achieving one or more clinical objectives, including achieving at least a minimum dose to a target volume and minimizing a dose to an organ at risk;
minimizing the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix; and
updating the set of leaf positions of the VMAT treatment plan according to the proposed leaf positions of the minimized EOF.

2. The method of claim 1, wherein the minimizing and updating steps are performed for each control point of the VMAT treatment plan.

3. The method of claim 1, wherein the one or more clinical objectives of the EOF are different from clinical objectives used to generate the VMAT treatment plan.

4. The method of claim 1, wherein the beamlet dose matrices are calculated using Monte Carlo routines.

5. The method of claim 1, wherein the proposed leaf position of each leaf is represented by a vector (x) of ternary leaf variables, and the EOF (ƒ_{E}) is a function of the vector (ƒ_{E}(x)).

6. The method of claim 5, wherein x=[x₁, x₂, . . . , xₙ], where n is the number of active leaves in the VMAT treatment plan and xᵢ∈{−1, 0, 1}, where i is an index value, −1 is a move of 1 unit distance into the field, 1 is a move of 1 unit distance out of the field, and 0 is an unchanged leaf position.

7. The method of claim 6, wherein the EOF is ƒ_{E}(x)=ΣⱼΣᵢWⱼ[(d_{j,min}−dᵢ(x))²*H{d_{j,min}−dᵢ(x)}+(dᵢ(x)−d_{j,max})²*H{dᵢ(x)−d_{j,max}}], where j is an index of clinical objectives, d_{j,min} is a minimum-dose objective, d_{j,max} is a maximum-dose objective, i is a voxel, Wⱼ is a weight for each clinical objective, and H is either 1 or 0 to eliminate terms which do not violate the clinical objective.

8. The method of claim 1, further comprising recalculating the updated VMAT treatment plan with linac and/or leaf-motion constraints.

9. The method of claim 8, further comprising generating dose-volume histograms and/or isodose curves of the updated VMAT treatment plan.

10. A VMAT treatment plan optimization system, comprising:
a processor; and
a memory in electronic communication with the processor, the memory comprising instructions for the processor to:
obtain a VMAT treatment plan from a treatment planning system (TPS), the VMAT treatment plan having a plurality of control points, each control point having a set of leaf positions corresponding a set of leaves of a multileaf collimator (MLC) in a field of a linear accelerator (linac);
calculate a radiation dose matrix corresponding to each beamlet, wherein a beamlet is the change in field when an MLC leaf is moved a predetermined unit distance;
define an enhanced objective function (EOF) for achieving one or more clinical objectives, including achieving at least a minimum dose to a target volume and minimizing a dose to an organ at risk;
minimize the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix;
and
update the set of leaf positions of the VMAT treatment plan according to the proposed leaf positions of the minimized EOF.

11. The system of claim 10, wherein the processor performs the minimizing and updating steps for each control point of the VMAT treatment plan.

12. The system of claim 10, wherein the one or more clinical objectives of the EOF are different from clinical objectives used to generate the VMAT treatment plan.

13. The system of claim 10, wherein the processor calculates the beamlet dose matrices using Monte Carlo routines.

14. The system of claim 10, wherein the proposed leaf position of each leaf is represented by a vector (x) of ternary leaf variables, and the EOF (ƒ_{E}) is a function of the vector (ƒ_{E}(x)).

15. The system of claim 14, wherein x=[x₁, x₂, . . . , xₙ], where n is the number of active leaves in the VMAT treatment plan and xᵢ∈{−1, 0, 1}, where i is an index value, −1 is a move of 1 unit distance into the field, 1 is a move of 1 unit distance out of the field, and 0 is an unchanged leaf position.

16. The system of claim 15, wherein the EOF is ƒ_{E}(x)=ΣⱼΣᵢWⱼ[(d_{j,min}−dᵢ(x))²*H{d_{j,min}−dᵢ(x)}+(dᵢ(x)−d_{j,max})²*H{dᵢ(x)−d_{j,max}}], where j is an index of clinical objectives, d_{j,min} is a minimum-dose objective, d_{j,max} is a maximum-dose objective, i is a voxel, Wⱼ is a weight for each clinical objective, and H is either 1 or 0 to eliminate terms which do not violate the clinical objective.

17. The system of claim 10, wherein the processor is further instructed to recalculate the updated VMAT treatment plan with linac and/or leaf-motion constraints.

18. The system of claim 17, wherein the processor is further instructed to generate dose-volume histograms and/or isodose curves of the updated VMAT treatment plan.

19. A non-transitory computer-readable medium encoded with computer-executable instructions, which when executed by a processor, cause the processor to:
obtain a VMAT treatment plan from a treatment planning system (TPS), the VMAT treatment plan having a plurality of control points, each control point having a set of leaf positions corresponding a set of leaves of a multileaf collimator (MLC) in a field of a linear accelerator (linac);
calculate a radiation dose matrix corresponding to each beamlet, wherein a beamlet is the change in field when an MLC leaf is moved a predetermined unit distance;
define an enhanced objective function (EOF) for achieving one or more clinical objectives, including achieving at least a minimum dose to a target volume and minimizing a dose to an organ at risk;
minimize the EOF for proposed leaf positions iterating through each leaf of at least a subset of the leaves of the VMAT treatment plan, wherein the proposed leaf positions move each leaf into the field or out of the field by the predetermined unit distance and corresponds to the addition or subtraction of the corresponding radiation dose matrix; and
update the set of leaf positions of the VMAT treatment plan according to the proposed leaf positions of the minimized EOF.

20. A method for optimizing a volumetric modulated arc therapy (VMAT) treatment plan, comprising:
obtaining a VMAT treatment plan from a treatment planning system (TPS), the VMAT treatment plan having a plurality of control points, each control point having a weight corresponding to an intensity of the linear accelerator (linac) beam at the associated control point;
defining an enhanced objective function (EOF) for achieving one or more clinical objectives, including achieving at least a minimum dose to a target volume and minimizing a dose to an organ at risk;
calculating a radiation dose matrix associated with each control point;
minimizing the EOF iterating through a varying weight of each control point, which corresponds to increasing or decreasing the associated radiation dose matrix by a scale factor; and
updating the weight of each control point of the VMAT treatment plan according to the minimized EOF.

21. The method of claim 20, wherein the dose matrix for each control point is calculated using a monte carlo (MC) dose calculation.

22. The method of claim 20, wherein the one or more clinical objectives of the EOF are different from clinical objectives used to generate the VMAT treatment plan.

23. The method of claim 20, wherein the minimization step is performed using a continuous optimization routine.

24. The method of claim 20, wherein the weight is varied from 0 to a predetermined maximum weight.

25. A VMAT treatment plan optimization system, comprising:
a processor; and
a memory in electronic communication with the processor, the memory comprising instructions for the processor to:
obtain an optimized VMAT treatment plan from a treatment planning system (TPS), the VMAT treatment plan having a plurality of control points, each control point having a weight corresponding to an intensity of the linear accelerator (linac) beam at the associated control point;
define an enhanced objective function (EOF) for achieving one or more clinical objectives, including achieving at least a minimum dose to a target volume and minimizing a dose to an organ at risk;
calculate a radiation dose matrix associated with each control point;
minimize the EOF iterating through a varying weight of each control point, which corresponds to increasing or decreasing of the associated dose matrix by a scale factor; and
update the weight of each control point of the VMAT treatment plan according to the minimized EOF.

26. The system of claim 25, wherein the dose matrix for each control point is calculated using a monte carlo (MC) dose calculation.

27. The system of claim 25, wherein the one or more clinical objectives of the EOF are different from clinical objectives used to generate the VMAT treatment plan.

28. The system of claim 25, wherein the minimization step is performed using a continuous optimization routine.

29. The system of claim 25, wherein the weight is varied from 0 to a predetermined maximum weight.

30. A non-transitory computer-readable medium encoded with computer-executable instructions, which when executed by a processor, cause the processor to:
obtain an optimized VMAT treatment plan from a treatment planning system (TPS), the VMAT treatment plan having a plurality of control points, each control point having a weight corresponding to an intensity of the linear accelerator (linac) beam at the associated control point;
define an enhanced objective function (EOF) for achieving one or more clinical objectives, including achieving at least a minimum dose to a target volume and minimizing dose to one or more organs at risk;
calculate a dose matrix associated with each control point;
minimize the EOF iterating through a varying weight of each control point, which corresponds to increasing or decreasing of the associated dose matrix by a scale factor; and
update the weight of each control point of the VMAT treatment plan according to the minimized EOF.
